(19) United States
(12) Patent Application Publication
(10) Pub. No.: US 2021/0364918 A1
(43) Pub. Date: Nov. 25, 2021

(54) RADIATION-SENSITIVE RESIN COMPOSITION AND METHOD FOR FORMING RESIST PATTERN

(71) Applicant: JSR CORPORATION, Tokyo (JP)

(72) Inventors: Katsuaki Nishikori, Tokyo (JP); Shuto Mori, Tokyo (JP); Junya Suzuki, Tokyo (JP); Hiromitsu Nakashima, Tokyo (JP)

(73) Assignee: JSR CORPORATION, Tokyo (JP)

(21) Appl. No.: 17/392,435

(22) Filed: Aug. 3, 2021

Related U.S. Application Data
(63) Continuation-in-part of application No. PCT/JP2020/007073, filed on Feb. 21, 2020.

(30) Foreign Application Priority Data
Mar. 28, 2019 (JP) 2019-064059

(51) Int. Cl. G03F 7/004; C08F 220/18; C07C 381/12; C07C 65/05

(52) U.S. Cl. CPC G03F 7/0045; C08F 220/1806; C08F 220/1807; C08F 220/1808; C07C 65/05; C08F 220/1805; C08F 220/1809; C07C 381/12; C08F 220/1812

(57) ABSTRACT

A radiation-sensitive resin composition contains a resin including a structural unit having a phenolic hydroxyl group; and a compound represented by formula (1). In the formula (1), Ar is a substituted or unsubstituted aromatic ring having 6 to 20 carbon atoms; n is an integer of 2 to 4; Z⁺ is a monovalent onium cation; a plurality of Ys are each independently a polar group; and at least one of the plurality of Ys is an —OH group or an —SH group bonded to a carbon atom adjacent to a carbon atom to which a COO⁻ group is bonded.

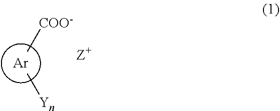

### CROSS-REFERENCE TO RELATED APPLICATIONS

The present application is a continuation-in-part application of International Application No. PCT/JP2020/007073, filed Feb. 21, 2020, which claims priority to Japanese Patent Application No. 2019-064059 filed Mar. 28, 2019. The contents of these applications are incorporated herein by reference in their entirety.

### BACKGROUND OF THE INVENTION

### Field of the Invention

The present invention relates to a radiation-sensitive resin composition and a method for forming a resist pattern.

### Description of the Related Art

A photolithography technology using a resist composition has been used for the fine circuit formation in a semiconductor device. As the representative procedure, for example, a resist pattern is formed on a substrate by generating an acid by irradiating the coating of the resist composition with a radioactive ray through a mask pattern, and then reacting in the presence of the acid as a catalyst to generate the difference of solubility of a resin into an alkaline or organic developer between an exposed part and a non-exposed part.

In the photolithography technique, the micronization of the pattern is promoted by using shorter wavelength radiations such as ArF excimer laser, and by using immersion exposure method (liquid immersion lithography) in which exposure is carried out in a state that a space between a lens of an exposing apparatus and a resist film is filled with liquid medium.

While efforts for further technique evolution are in progress, a technique has been proposed, in which a photosensitive quencher is blended in a resist composition, and an acid diffused to an unexposed portion is captured by an ion exchange reaction to improve lithography performance by ArF exposure (JP-A-2013-200560).

### SUMMARY OF THE INVENTION

According to an aspect of the present invention, a radiation-sensitive resin composition includes: a resin including a structural unit having a phenolic hydroxyl group; and a compound represented by formula (1).

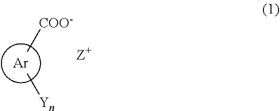

In the formula (1): Ar is a substituted or unsubstituted aromatic ring having 6 to 20 carbon atoms; n is an integer of 2 to 4; Z⁺ is a monovalent onium cation; a plurality of Ys are each independently a polar group; and at least one of the plurality of Ys is an —OH group or an —SH group bonded to a carbon atom adjacent to a carbon atom to which a COO group is bonded.

According to another aspect of the present invention, a method for forming a resist pattern includes forming a resist film directly or indirectly on a substrate by applying the above-mentioned radiation-sensitive resin composition. The resist film is exposed, and the exposed resist film is developed.

According to a further aspect of the present invention, a radiation-sensitive resin composition includes: a resin including a structural unit having an acid-dissociable group and not including a structural unit having a phenolic hydroxyl group; a compound represented by formula (1); and a radiation-sensitive acid generator which generates an acid having a pKa smaller than that of an acid generated from the compound. A content of the radiation-sensitive acid generator is 10 parts by mass or more with respect to 100 parts by mass of the resin.

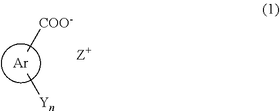

In the formula (1): Ar is a substituted or unsubstituted aromatic ring having 6 to 20 carbon atoms; n is an integer of 2 to 4; Z⁺ is a monovalent onium cation; a plurality of Ys are each independently a polar group; and at least one of the plurality of Ys is an —OH group or an —SH group bonded to a carbon atom adjacent to a carbon atom to which a COO⁻ group is bonded.

According to a further aspect of the present invention, a method for forming a resist pattern includes forming a resist film directly or indirectly on a substrate by applying the above-mentioned radiation-sensitive resin composition. The resist film is exposed, and the exposed resist film is developed.

### DETAILED DESCRIPTION OF THE PREFERRED EMBODIMENTS

As a next-generation exposure technique, lithography using shorter wavelength radiations such as electron beam, X ray and extreme ultraviolet ray (EUV) has also been studied. In the above-described next-generation exposure technique, various resist performances such as sensitivity and depth of focus equal to or higher than ever before are required, and process margins for facilitating control of conditions for pattern miniaturization are desired.

That is, the present invention relates, in one embodiment, to a radiation-sensitive resin composition containing:

a resin including a structural unit having a phenolic hydroxyl group; and

a compound represented by the following formula (1):

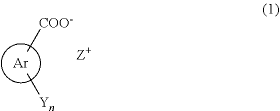

in the formula (1), Ar is a substituted or unsubstituted aromatic ring having 6 to 20 carbon atoms; n is an integer of 2 to 4; Z⁺ is a monovalent onium cation; a plurality of Ys are each independently a polar group; and at least one of the plurality of Ys is an —OH group or an —SH group bonded to a carbon atom adjacent to a carbon atom to which a COO⁻ group is bonded.

In the radiation-sensitive resin composition, the compound represented by the above formula (1) having a specific structure (hereinafter, also referred to as “compound (B)”) is blended as a quencher, whereby excellent sensitivity, depth of focus, and process margins can be exhibited. The reason for this is not bound by any theory, but is presumed as follows. When an exposure amount is less than a required amount (in the case of underdose), the deprotection of an acid-dissociable group of a resin becomes insufficient, whereby the solubility of an exposed portion in a developer tends to be deteriorated, which may cause bridge defects or scum occurrence. By introducing a plurality of polar groups into the compound (B) to increase the polarity of the compound (B), the solubility of the compound (B) in the alkaline developer in a portion where the exposure amount is insufficient is increased, whereby the above problems can be suppressed. When the pattern cross-sectional shape of the exposed portion is a T shape or a reverse wedge shape (a state where the bottom portion is thinner than the upper portion) during defocus, pattern collapse is apt to occur. Since the thick portion of the upper portion of the pattern is considered to be an insufficiently deprotected portion, the solubility of the upper portion of the pattern is enhanced by increasing the polarity of the compound (B) as in the case of the underexposure amount, whereby the above problems are suppressed. As described above, it is presumed that the radiation-sensitive resin composition can exhibit sensitivity, depth of focus, and process margins at adequate levels by blending the compound (B) and by compensating for insufficient solubility in the exposed portion during a low exposure amount or defocus.

In one embodiment, the polar group bonded to the carbon atom adjacent to the carbon atom to which the COO⁻ group is bonded is preferably an —OH group. By adopting the —OH group as the polar group, the polarity of the compound (B) can be further enhanced, whereby sensitivity, depth of focus, and process margins can be exhibited at higher levels.

In one embodiment, the compound represented by the formula (1) is preferably a compound represented by the following formula (1-1):

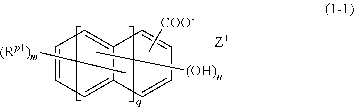

in the formula (1-1), R^{p1} is an alkoxy group, an alkoxycarbonyl group, a halogen atom, or an amino group; m is an integer of 0 to 3; when m is 2 or 3, a plurality of R^{p1}s are the same or different; n and Z⁺ have the same meanings as those in the above formula (1); q is an integer of 0 to 2; when q is 0, m+n is less than or equal to 5; and at least one OH group is bonded to the carbon atom adjacent to the carbon atom to which the COO⁻ group is bonded.

By adopting the compound represented by the formula (1-1) (hereinafter, also referred to as “compound (b)”) as the compound (B), the polarity of the compound (B) can be efficiently enhanced, whereby the sensitivity, the depth of focus, and the process margins can be efficiently improved.

In one embodiment, q in the formula (1-1) is preferably 0 or 1. This makes it possible to enhance the solubility of the compound (b) in the alkaline developer while maintaining affinity with a resin (A).

In one embodiment, n in the formula (1-1) is preferably 2 or 3. Thereby, the polarity and stability and the like of the compound (b) can be enhanced.

In one embodiment, the content of the compound represented by the formula (1) is preferably 3 parts by mass or more and 30 parts by mass or less with respect to 100 parts by mass of the resin. Thereby, the solubility improving action of the compound (B) can be obtained at adequate levels, whereby the sensitivity, the depth of focus, and the process margins can be exhibited at higher levels.

In one embodiment, the onium cation in the formula (1) is preferably a sulfonium cation or an iodonium cation.

In one embodiment, the radiation-sensitive resin composition preferably further contains a radiation-sensitive acid generator which generates an acid having a pKa smaller than that of an acid generated from the compound represented by the formula (1). When the radiation-sensitive resin composition specifically contains a radiation-sensitive acid generator, a protecting group in the resin (A) can be deprotected, whereby a lithographic process can be suitably advanced.

In one embodiment, a content of the radiation-sensitive acid generator is preferably 10 parts by mass or more with respect to 100 parts by mass of the resin. A content of the radiation-sensitive acid generator is preferably 10 parts by mass or more and 60 parts by mass or less with respect to 100 parts by mass of the resin. As a result, the sensitivity, the depth of focus, and the process margins can be further improved.

In one embodiment, the structural unit having a phenolic hydroxyl group is preferably a structural unit derived from hydroxystyrene. In the case of adopting exposure by EUV or the like, light absorption by a base resin, which has caused a problem in conventional exposure by ArF excimer laser light or the like, does not cause a problem, whereby a structural unit derived from hydroxystyrene having high etching resistance can be efficiently introduced.

In one embodiment, a content ratio of the structural unit having a phenolic hydroxyl group in the resin is preferably 5 mol % or more and 70 mol % or less. Thereby, the etching resistance of the obtained pattern can be further improved.

The present invention relates, in another embodiment, to a method for forming a resist pattern including: forming a resist film from the radiation-sensitive resin composition; exposing the resist film; and developing the exposed resist film.

In the method for forming a resist pattern, the radiation-sensitive resin composition having excellent sensitivity, depth of focus, and process margins is used, whereby a high-quality resist pattern can be efficiently formed.

In another embodiment, by adopting the radiation-sensitive resin composition having excellent sensitivity, depth of focus, and process margins, the exposure can be suitably performed using extreme ultraviolet ray or electron beam, whereby a desired fine pattern can be efficiently formed.

The present invention relates, in still another embodiment, to a radiation-sensitive resin composition containing: a resin including a structural unit having an acid-dissociable group and not containing a structural unit having a phenolic hydroxyl group; a compound represented by the following formula (1); and a radiation-sensitive acid generator which generates an acid having a pKa smaller than that of an acid generated from the compound, wherein a content of the radiation-sensitive acid generator is 10 parts by mass or more with respect to 100 parts by mass of the resin.

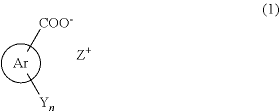

in the formula (1), Ar is a substituted or unsubstituted aromatic ring having 6 to 20 carbon atoms; n is an integer of 2 to 4; Z⁺ is a monovalent onium cation; a plurality of Ys are each independently a polar group; and at least one of the plurality of Ys is an —OH group or an —SH group bonded to a carbon atom adjacent to a carbon atom to which a COO group is bonded.

Hereinafter, embodiments of the present invention will be described in detail, but the present invention is not limited to these embodiments.

### First Embodiment

### <Radiation-Sensitive Resin Composition>

A radiation-sensitive resin composition according to the present embodiment (hereinafter, also simply referred to as “composition”) contains a resin (A) and a compound (B). The radiation-sensitive resin composition further contains a radiation-sensitive acid generator (C) and a solvent (D) if necessary. The composition may contain other optional components as long as the effects of the present invention are not impaired.

### (Resin (A))

The resin (A) is an assembly of polymers having a structural unit (a1) having a phenolic hydroxyl group (hereinafter, this resin is also referred to as “base resin”). The resin (A) as the base resin may have, in addition to the structural unit (a1), a structural unit (a2) having an acid-dissociable group or other structural units. Each structural unit will be described below.

[Structural Unit (a1)]

The structural unit (a1) is a structural unit including a phenolic hydroxyl group. The resin (A) has the structural unit (a1) and other structural units if necessary, whereby the solubility of the resin (A) in a developer can be more appropriately adjusted, and as a result, the sensitivity and the like of the radiation-sensitive resin composition can be further improved. When KrF excimer laser light, EUV, or electron beam or the like is used as radiation to be irradiated in an exposuring in a method for forming a resist pattern, the resin (A) has the structural unit (a1), whereby the structural unit (a1) contributes to improvement in etching resistance and improvement in a difference (dissolution contrast) in solubility to a developer between an exposed portion and an unexposed portion. In particular, the resin (A) can be suitably applied to pattern formation using exposure with radiation having a wavelength of 50 nm or less such as electron beam or EUV.

In the radiation-sensitive resin composition of the present embodiment, the structural unit (a1) may be a structural unit derived from hydroxystyrene.

Examples of the structural unit (a1) include a structural unit represented by the following formula (af).

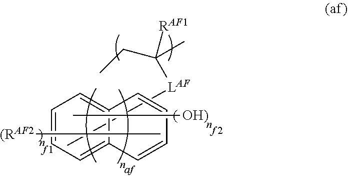

In the above formula (af), R^{AF1} is a hydrogen atom or a methyl group. L^{AF} is a single bond, —COO—, —O—, or —CONH—. R^{AF2} is a monovalent organic group having 1 to 20 carbon atoms. n_{f1} is an integer of 0 to 3. When n_{f1} is 2 or 3, a plurality of R^{AF2}s may be the same or different. n_{f2} is an integer of 1 to 3. However, n_{f1}+n_{f2} is 5 or less. n_{af} is an integer of 0 to 2.

R^{AF1} is preferably a hydrogen atom from the viewpoint of the copolymerizability of a monomer giving the structural unit (a1).

L^{AF} is preferably a single bond or —COO—.

The organic group in the resin (A) refers to a group including at least one carbon atom.

Examples of a monovalent organic group having 1 to 20 carbon atoms, represented by R^{AF2} include a monovalent hydrocarbon group having 1 to 20 carbon atoms, a group containing a divalent hetero atom-containing group between two adjacent carbon atoms or at the end of the atomic bonding side of the hydrocarbon group, and a group obtained by substituting with a monovalent hetero atom-containing group, a part or all of hydrogen atoms contained in the group and the hydrocarbon group.

Examples of the monovalent hydrocarbon group having 1 to 20 carbon atoms, represented by R^{AF2} include:

alkyl groups such as a methyl group, an ethyl group, a propyl group, and a butyl group;

alkenyl groups such as an ethenyl group, a propenyl group, and a butenyl group;

chain hydrocarbon groups such as alkynyl groups (such as an ethynyl group, a propynyl group, and a butynyl group);

cycloalkyl groups such as a cyclopropyl group, a cyclopentyl group, a cyclohexyl group, a cyclooctyl group, a norbornyl group, and an adamantyl group;

alicyclic hydrocarbon groups such as cycloalkenyl groups (such as a cyclopropenyl group, a cyclopentenyl group, a cyclohexenyl group, and a norbornenyl group);

aryl groups such as a phenyl group, a tolyl group, a xylyl group, a naphthyl group, and an anthryl group; and

aromatic hydrocarbon groups such as aralkyl groups (such as a benzyl group, a phenethyl group, and a naphthylmethyl group).

The R^{AF2} is preferably a chain hydrocarbon group or a cycloalkyl group, more preferably an alkyl group and a cycloalkyl group, and still more preferably a methyl group, an ethyl group, a propyl group, a cyclopentyl group, a cyclohexyl group, a cyclooctyl group, and an adamantyl group.

Examples of the divalent heteroatom-containing group include —O—, —CO—, —CO—O—, —S—, —CS—, —SO₂—, —NR′—, and a group obtained by combining two or more of these. R′ is a hydrogen atom or a monovalent hydrocarbon group.

Examples of the monovalent heteroatom-containing group include halogen atoms such as a fluorine atom, a chlorine atom, a bromine atom, and an iodine atom, a hydroxy group, a carboxy group, a cyano group, an amino group, and a sulfanyl group (—SH).

Among them, a monovalent chain hydrocarbon group is preferable; an alkyl group is more preferable; and a methyl group is still more preferable.

n_{f1} is preferably an integer of 0 to 2, more preferably 0 and 1, and still more preferably 0.

n_{f2} is preferably 1 or 2, and more preferably 1.

n_{af} is preferably 0 and 1, and more preferably 0.

The structural unit (a1) is preferably a structural unit represented by each of the following formulae (a1-1) to (a1-6), or the like.

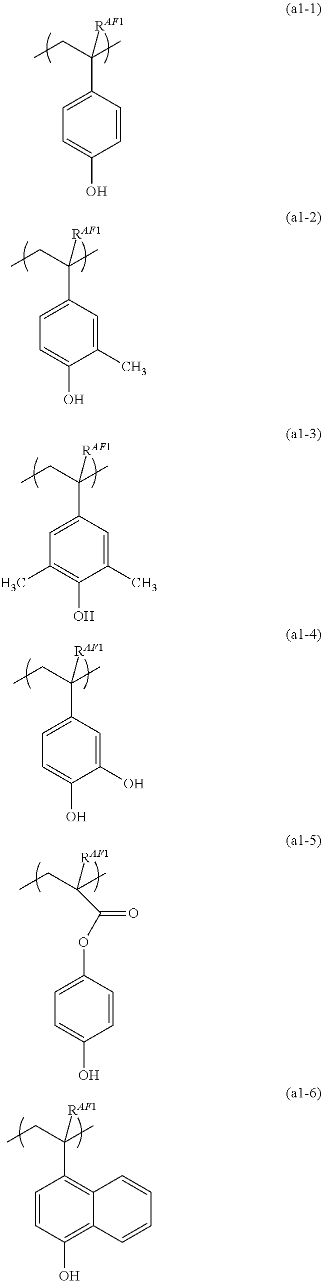

In the formulae (a1-1) to (a1-6), R^{AF1} is the same as that in the formula (af).

Among them, the structural units (a1-1) and (a1-2) are preferable, and the structural unit (a1-1) is more preferable.

The lower limit of the content ratio of the structural unit (a1) in the resin (A) is preferably 5 mol %, more preferably 10 mol %, still more preferably 15 mol %, and particularly preferably 20 mol %, with respect to the total structural units constituting the resin (A). The upper limit of the content ratio is preferably 70 mol %, more preferably 60 mol %, still more preferably 55 mol %, and particularly preferably 50 mol %. By setting the content ratio of the structural unit (a1) within the above range, the sensitivity, depth of focus, and process margins of the radiation-sensitive resin composition can be further improved.

When a monomer having a phenolic hydroxyl group such as hydroxystyrene is directly radically polymerized, the polymerization may be inhibited under the influence of the phenolic hydroxyl group. In this case, it is preferable that the structural unit (a1) is obtained by polymerizing the monomer in a state where the phenolic hydroxyl group is protected by a protecting group such as an alkali-dissociable group, and then deprotecting the polymerized product by hydrolysis. The structural unit which provides the structural unit (a1) by hydrolysis is preferably represented by the following formula (4).

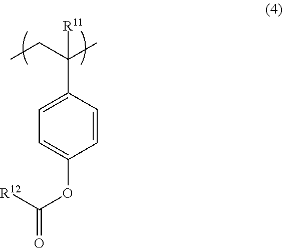

In the formula (4), R¹¹ is a hydrogen atom, a fluorine atom, a methyl group, or a trifluoromethyl group. R¹² is a monovalent hydrocarbon group having 1 to 20 carbon atoms or an alkoxy group. Examples of the monovalent hydrocarbon group having 1 to 20 carbon atoms of R¹² include a monovalent chain hydrocarbon group having 1 to 20 carbon atoms. Examples of the alkoxy group include a methoxy group, an ethoxy group, and a tert-butoxy group.

As R¹², an alkyl group and an alkoxy group are preferable, and a methyl group and a tert-butoxy group are more preferable.

[Structural Unit (a2)]

The structural unit (a2) is a structural unit including an acid-dissociable group. Among them, the acid-dissociable group in the structural unit (a2) preferably contains a cyclic structure. Examples of the acid-dissociable group having a cyclic structure include a structural unit having a tertiary alkyl ester moiety, a structural unit having a structure in which a hydrogen atom of a phenolic hydroxyl group is substituted with a tertiary alkyl group, and a structural unit having an acetal bond. From the viewpoint of improving the patternability of the radiation-sensitive resin composition, a structural unit represented by the following formula (5) (hereinafter, also referred to as “structural unit (a2-1)”) is preferable.

In the present invention, the “acid-dissociable group” refers to a group which substitutes a hydrogen atom of a carboxy group, a phenolic hydroxyl group, an alcoholic hydroxyl group, or a sulfo group or the like, and is dissociated by the action of an acid. The radiation-sensitive resin composition has excellent patternability because the resin has the structural unit (a2).

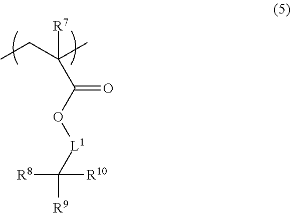

In the formula (5), R⁷ is a hydrogen atom, a fluorine atom, a methyl group, or a trifluoromethyl group. R⁸ is a hydrogen atom or a monovalent hydrocarbon group having 1 to 20 carbon atoms. R⁹ and R¹⁰ each independently represent a monovalent chain hydrocarbon group having 1 to 10 carbon atoms or a monovalent alicyclic hydrocarbon group having 3 to 20 carbon atoms, or represent a divalent alicyclic group having 3 to 20 carbon atoms, composed of these groups combined with carbon atoms to which the groups are bonded. One of R⁸ to R¹⁰ has at least one or more cyclic structures or a plurality of R⁸ to R¹⁰ are combined with each other to have at least one or more cyclic structures. L¹ represents a single bond or a divalent linking group.

R⁷ is preferably a hydrogen atom or a methyl group, and more preferably a methyl group, from the viewpoint of the copolymerizability of the monomer giving the structural unit (a2-1).

Examples of the monovalent hydrocarbon group having 1 to 20 carbon atoms, represented by R⁸ include a chain hydrocarbon group having 1 to 10 carbon atoms, a monovalent alicyclic hydrocarbon group having 3 to 20 carbon atoms, and a monovalent aromatic hydrocarbon group having 6 to 20 carbon atoms.

Examples of the chain hydrocarbon group having 1 to 10 carbon atoms, represented by R⁸ to R¹⁰ include a linear or branched saturated hydrocarbon group having 1 to 10 carbon atoms or a linear or branched unsaturated hydrocarbon group having 1 to 10 carbon atoms.

Examples of the alicyclic hydrocarbon group having 3 to 20 carbon atoms, represented by R⁸ to R¹⁰ include a monocyclic or polycyclic saturated hydrocarbon group, or a monocyclic or polycyclic unsaturated hydrocarbon group. The monocyclic saturated hydrocarbon group is preferably a cyclopentyl group, a cyclohexyl group, a cycloheptyl group, or a cyclooctyl group. The monocyclic unsaturated hydrocarbon group is preferably a cyclopentenyl group, a cyclohexenyl group, a cycloheptenyl group, or a cyclooctenyl group. The polycyclic cycloalkyl group is preferably a bridged alicyclic hydrocarbon group such as a norbornyl group, an adamantyl group, a tricyclodecyl group, or a tetracyclododecyl group. The bridged alicyclic hydrocarbon group refers to a polycyclic alicyclic hydrocarbon group in which two carbon atoms which are not adjacent to each other among carbon atoms constituting an alicyclic ring are bonded by a bond chain containing one or more carbon atoms.

Examples of the monovalent aromatic hydrocarbon group having 6 to 20 carbon atoms, represented by R⁸ include aryl groups such as a phenyl group, a tolyl group, a xylyl group, a naphthyl group, and an anthryl group; and aralkyl groups such as a benzyl group, a phenethyl group, and a naphthylmethyl group.

R⁸ is preferably a linear or branched saturated hydrocarbon group having 1 to 10 carbon atoms or an alicyclic hydrocarbon group having 3 to 20 carbon atoms.

When any two or more of R⁸ to R¹⁰ are combined with each other to have at least one or more cyclic structures, the divalent alicyclic group having 3 to 20 carbon atoms formed by R⁹ and R¹⁰ combined together and a carbon atom to which a chain hydrocarbon group or an alicyclic hydrocarbon group represented by R⁹ and a chain hydrocarbon group or an alicyclic hydrocarbon group represented by R¹⁰ are bonded is not particularly limited as long as it is a group obtained by removing two hydrogen atoms from the same carbon atom constituting a carbon ring of a monocyclic or polycyclic alicyclic hydrocarbon having the above-described carbon number. The divalent alicyclic group may be either a monocyclic hydrocarbon group or a polycyclic hydrocarbon group. The polycyclic hydrocarbon group may be either a bridged alicyclic hydrocarbon group or a condensed alicyclic hydrocarbon group. The divalent alicyclic group may be either a saturated hydrocarbon group or an unsaturated hydrocarbon group. It is to be noted that the condensed alicyclic hydrocarbon group refers to a polycyclic alicyclic hydrocarbon group in which two or more alicyclic rings share their sides (bond between adjacent two carbon atoms).

When the monocyclic alicyclic hydrocarbon group is a saturated hydrocarbon group, preferred examples thereof include a cyclopentanediyl group, a cyclohexanediyl group, a cycloheptanediyl group, and a cyclooctanediyl group. When the monocyclic alicyclic hydrocarbon group is an unsaturated hydrocarbon group, preferred examples thereof include a cyclopentenediyl group, a cyclohexenediyl group, a cycloheptenediyl group, a cyclooctenediyl group, and a cyclodecenediyl group. The polycyclic alicyclic hydrocarbon group is preferably a bridged alicyclic saturated hydrocarbon group, and preferred examples thereof include a bicyclo[2.2.1]heptane-2,2-diyl group (norbornane-2,2-diyl group), a bicyclo[2,2,2]octane-2,2-diyl group, and a tricyclo[3.3.1.1^{3,7}]decane-2,2-diyl group (adamantane-2,2-diyl group).

Examples of the divalent linking group represented by L¹ include an alkanediyl group, a cycloalkanediyl group, an alkenediyl group, *—R^{LA}O—, and —R^{LB}COO—(* represents a bond on an oxygen side). Some or all of the hydrogen atoms of these groups may be substituted with a halogen atom such as a fluorine atom or a chlorine atom, or a cyano group, or the like.

The alkanediyl group is preferably an alkanediyl group having 1 to 8 carbon atoms.

Examples of the cycloalkanediyl group include monocyclic cycloalkanediyl groups such as a cyclopentanediyl group and a cyclohexanediyl group; and polycyclic cycloalkanediyl groups such as a norbornanediyl group and an adamantanediyl group. The cycloalkanediyl group is preferably a cycloalkanediyl group having 5 to 12 carbon atoms.

Examples of the alkenediyl group include an ethenediyl group, a propenediyl group, and a butenediyl group. The alkenediyl group is preferably an alkenediyl group having 2 to 6 carbon atoms.

Examples of R^{LA} of *—R^{LA}O— include the alkanediyl group, the cycloalkanediyl group, and the alkenediyl group. Examples of R^{LB} of *—R^{LB}COO— include the alkanediyl group, the cycloalkanediyl group, the alkenediyl group, and the arenediyl group. Examples of the arenediyl group include a phenylene group, a tolylene group, and a naphthylene group. The arenediyl group is preferably an arenediyl group having 6 to 15 carbon atoms.

When the alicyclic group constituted together with R⁹ and R¹⁰ includes an unsaturated bond and L¹ is a single bond, R⁸ is preferably a hydrogen atom.

Examples of the structural unit (a2-1) include structural units represented by the following formulae (5-1) to (5-4) (hereinafter, also referred to as “structural units (a2-1-1) to (a2-1-4)”).

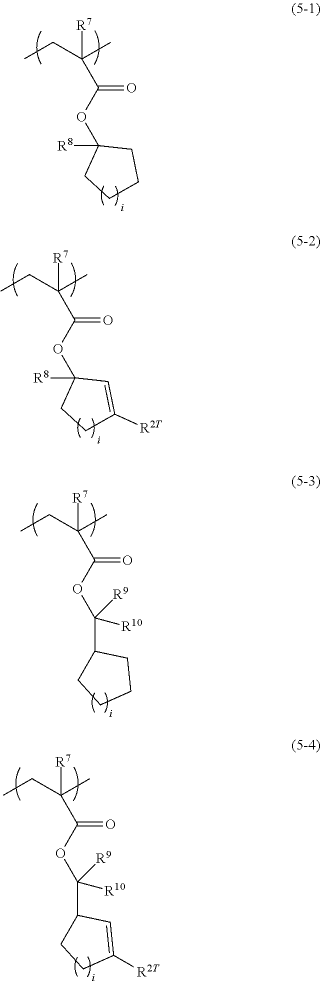

In the formula (5-1), R⁷ and R⁸ are the same as those in the formula (5). i is an integer of 1 to 4.

In the formula (5-2), R⁷ is the same as that in the formula (5). R⁸ is a hydrogen atom. R^{2T} is a hydrogen atom or a monovalent hydrocarbon group having 1 to 20 carbon atoms. i is an integer of 1 to 4.

In the above formulae (5-3) and (5-4), R⁷, R⁹, and R¹⁰ are the same as those in the above formula (5). R^{2T} is a hydrogen atom or a monovalent hydrocarbon group having 1 to 20 carbon atoms. i is an integer of 1 to 4.

As the structural unit (a2-1), among them, the structural unit (a2-1-1) and the structural unit (a2-1-2) are preferable, and a structural unit having a cyclopentane structure, a structural unit having a cyclohexane structure, a structural unit having a cyclopentene structure, and a structural unit having a cyclohexene structure are more preferable.

The resin (A) may contain one or a combination of two or more of the structural units (a2).

The lower limit of the content ratio of the structural unit (a2) is preferably 15 mol %, more preferably 20 mol %, still more preferably 25 mol %, and particularly preferably 30 mol % with respect to the total structural units constituting the resin (A) serving as the base resin. The upper limit of the content ratio is preferably 90 mol %, more preferably 80 mol %, still more preferably 75 mol %, and particularly preferably 70 mol %. By setting the content ratio of the structural unit (a2) within the above range, the patternability of the radiation-sensitive resin composition can be further improved.

(Structural Unit (a3))

The structural unit (a3) is a structural unit including at least one selected from the group consisting of a lactone structure, a cyclic carbonate structure and a sultone structure. The solubility of the base resin into a developer can be adjusted by further introducing the structural unit (a3). As a result, the radiation-sensitive resin composition can provide improved lithography properties such as the resolution. The adhesion between a resist pattern formed from the base resin and a substrate can also be improved.

Examples of the structural unit (a3) include structural units represented by the following formulae (T-1) to (T-10).

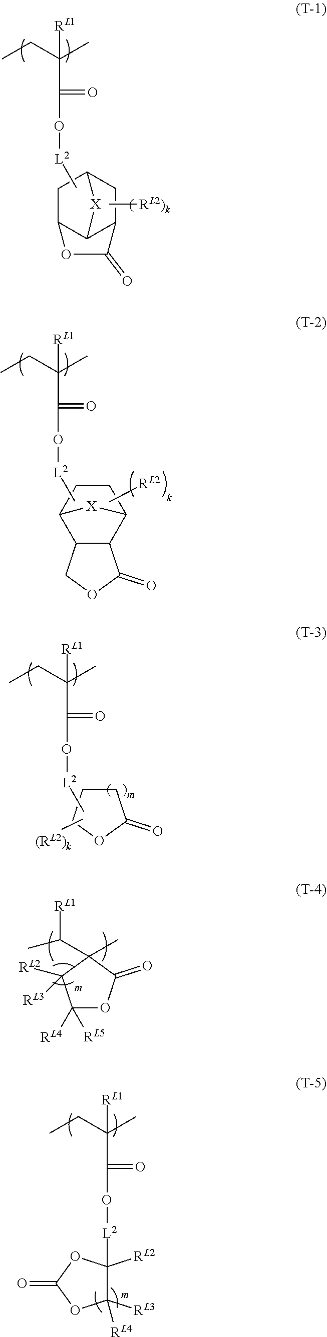

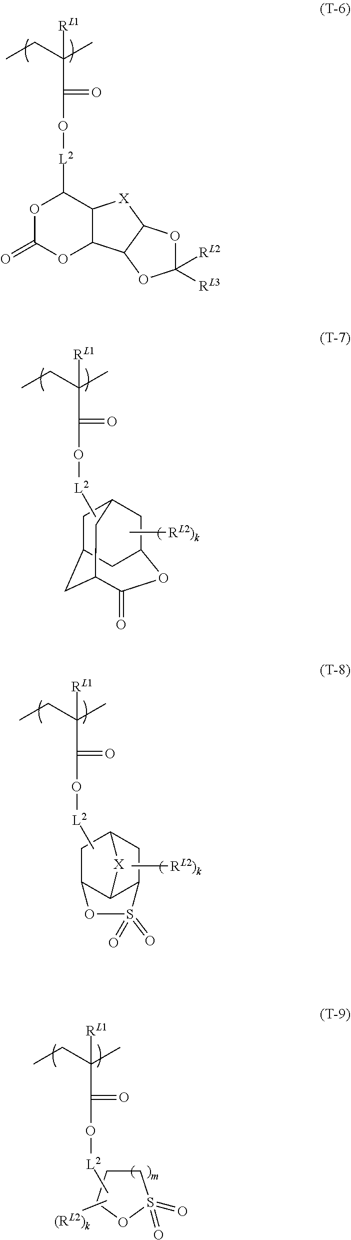

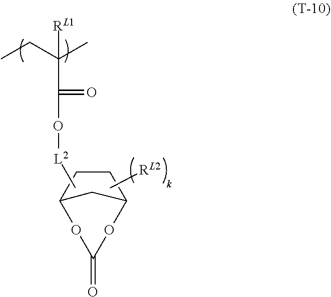

In the above formulae, R^{L1} is a hydrogen atom, a fluorine atom, a methyl group, or a trifluoromethyl group; R^{L2} to R^{L5} are each independently a hydrogen atom, an alkyl group having a carbon number of 1 to 4, a cyano group, a trifluoromethyl group, a methoxy group, a methoxycarbonyl group, a hydroxy group, a hydroxymethyl group, or a dimethylamino group; R^{L4} and R^{L5} may be a divalent alicyclic group having a carbon number of 3 to 8, which is obtained by combining R^{L4} and R^{L5} with the carbon atom to which they are bound. L² is a single bond, or a divalent linking group; X is an oxygen atom or a methylene group; k is an integer of 0 to 3; and m is an integer of 1 to 3.

Example of the divalent alicyclic group having a carbon number of 3 to 8, which is composed of a combination of R^{L4} and R^{L5} with the carbon atom to which they are bound, includes the divalent alicyclic group having a carbon number of 3 to 8 in the divalent alicyclic group having a carbon number of 3 to 20, which is composed of a combination of the chain hydrocarbon group or the alicyclic hydrocarbon group represented by R⁹ and R¹⁰ in the above formula (5) with the carbon atom to which they are bound. One or more hydrogen atoms on the alicyclic group may be substituted with a hydroxy group.

Examples of the divalent linking group represented by L² as described above include a divalent straight or branched chain hydrocarbon group having a carbon number of 1 to 10; a divalent alicyclic hydrocarbon group having a carbon number of 4 to 12; and a group composed of one or more of the hydrocarbon group thereof and at least one group of —CO—, —O—, —NH— and —S—.

Among them, the structural unit (a3) is preferably a group having a lactone structure, more preferably a group having a norbornane lactone structure, and further preferably a group derived from a norbornane lactone-yl (meth)acrylate.

The lower limit of the content by percent of the structural unit (a3) is preferably 5 mol %, more preferably 10 mol %, and further preferably 15 mol % based on the total structural units as the component of the base resin. The upper limit of the content by percent is preferably 40 mol %, more preferably 30 mol %, and further preferably 20 mol %. By adjusting the content by percent of the structural unit (II) within the ranges, the radiation-sensitive resin composition can provide improved lithography properties such as the resolution. The adhesion between the formed resist pattern and the substrate can also be improved.

[Structural Unit (a4)]

The resin (A) may appropriately have another structural unit (also referred to as structural unit (a4)) other than the structural units (a1) to (a3). Examples of the structural unit (a4) include a structural unit having a fluorine atom, an alcoholic hydroxyl group, a carboxy group, a cyano group, a nitro group, or a sulfonamide group or the like. Among them, a structural unit having a fluorine atom, a structural unit having an alcoholic hydroxyl group, and a structural unit having a carboxy group are preferable, and a structural unit having a fluorine atom and a structural unit having an alcoholic hydroxyl group are more preferable.

Examples of the structural unit (a4) include structural units represented by the following formulae.

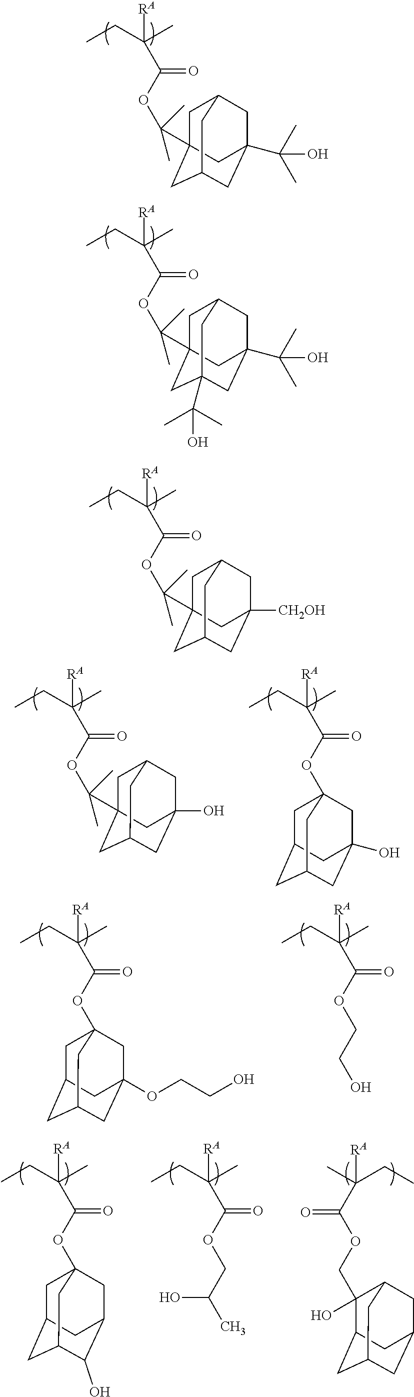

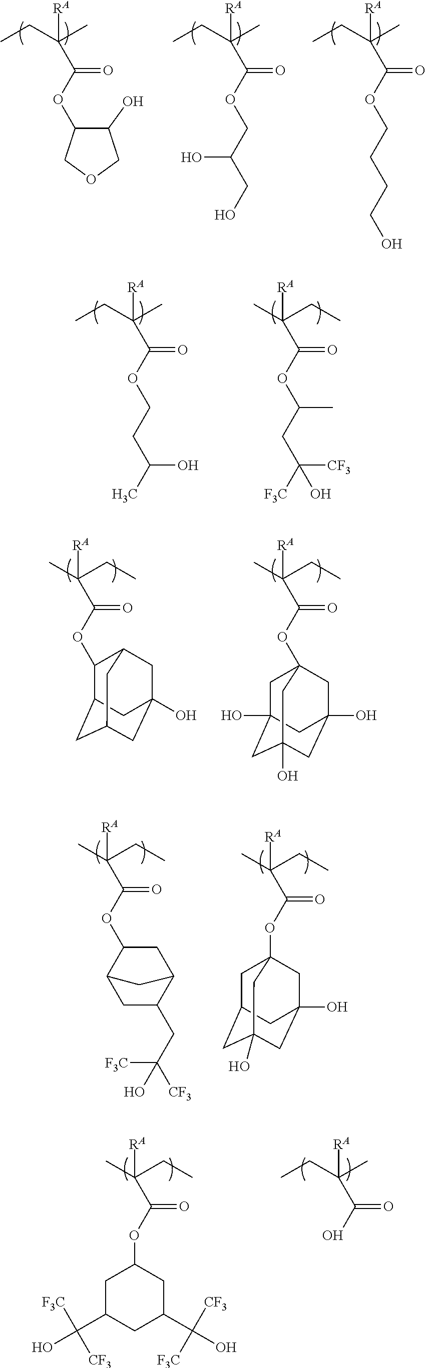

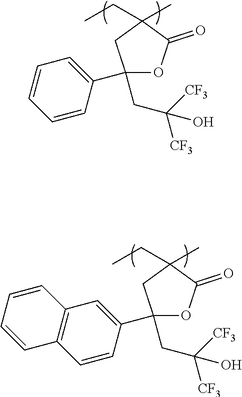

In the above formulae, R^{A} is a hydrogen atom, a fluorine atom, a methyl group, or a trifluoromethyl group.

When the resin (A) has the structural unit (a4), the lower limit of the content ratio of the structural unit (a4) to the total structural units constituting the resin (A) is preferably 1 mol %, more preferably 3 mol %, and still more preferably 5 mol %. Meanwhile, the upper limit of the content ratio is preferably 50 mol %, more preferably 40 mol %, and still more preferably 30 mol %. By setting the content ratios of the other structural units within the above range, the solubility of the resin (A) in the developer can be made more appropriate. When the content ratios of the other structural units are more than the above upper limit, the patternability may be deteriorated.

It is to be noted that the structural units (a2) to (a4) and other structural units do not include those falling under the structural unit (a1).

The content of the resin (A) is preferably 70 mass % or more, more preferably 75 mass % or more, even more preferably 80 mass % or more with respect to the total solid content of the radiation-sensitive resin composition. Here, the term “solid” refers to all components contained in the radiation-sensitive resin composition except for a solvent.

(Synthesis Method of Base Resin (A))

For example, the resin (A) as a base resin can be synthesized by performing a polymerization reaction of each monomer for providing each structural unit with a radical polymerization initiator or the like in a suitable solvent.

Examples of the radical polymerization initiator include an azo-based radical initiator, including azobisisobutyronitrile (AIBN), 2,2′-azobis(4-methoxy-2,4-dimethylvaleronitrile), 2,2′-azobis(2-cyclopropylpropanenitrile), 2,2′-azobis(2,4-dimethylvaleronitrile), and dimethyl 2,2′-azobisisobutyrate; and peroxide-based radical initiator, including benzoyl peroxide, t-butyl hydroperoxide, and cumene hydroperoxide. Among them, AIBN or dimethyl 2,2′-azobisisobutyrate is preferred, and AIBN is more preferred. The radical initiator may be used alone, or two or more radical initiators may be used in combination.

Examples of the solvent used for the polymerization reaction include

alkanes including n-pentane, n-hexane, n-heptane, n-octane, n-nonane, and n-decane;

cycloalkanes including cyclohexane, cycloheptane, cyclooctane, decalin, and norbornane;

aromatic hydrocarbons including benzene, toluene, xylene, ethylbenzene, and cumene;

halogenated hydrocarbons including chlorobutanes, bromohexanes, dichloroethanes, hexamethylenedibromide, and chlorobenzenes;

saturated carboxylate esters, including ethyl acetate, n-butyl acetate, i-butyl acetate, and methyl propionate;

ketones including acetone, methyl ethylketone, 4-methyl-2-pentanone, and 2-heptanone;

ethers including tetrahydrofuran, dimethoxyethanes, and diethoxyethanes; and

alcohols including methanol, ethanol, 1-propanol, 2-propanol, and 4-methyl-2-pentanol. The solvent used for the polymerization reaction may be used alone, or two or more solvents may be used in combination.

The reaction temperature of the polymerization reaction is typically from 40° C. to 150° C., and preferably from 50° C. to 120° C. The reaction time is typically from 1 hour to 48 hours, and preferably from 1 hour to 24 hours.

The molecular weight of the resin (A) as a base resin is not particularly limited, but the polystyrene-equivalent weight-average molecular weight (Mw) of the resin as measured by gel permeation chromatography (GPC) is preferably 1,000 or more and 50,000 or less, more preferably 2,000 or more and 30,000 or less, even more preferably 3,000 or more and 15,000 or less, particularly preferably 4,000 or more and 12,000 or less. If the Mw of the resin is less than the above lower limit, there is a case where the heat resistance of a resulting resist film is reduced. If the Mw of the resin exceeds the above upper limit, there is a case where the developability of a resist film is reduced.

For the base resin (A) as a base resin, the ratio of Mw to the number average molecular weight (Mn) as determined by GPC relative to standard polystyrene (Mw/Mn) is typically not less than 1 and not more than 5, preferably not less than 1 and not more than 3, and more preferably not less than 1 and not more than 2.

The Mw and Mn of the resin in the specification are amounts measured by using Gel Permeation Chromatography (GPC) with the condition as described below.

GPC column: two G2000HXL, one G3000HXL, and one G4000HXL (all manufactured from Tosoh Corporation)

Column temperature: 40° C.

Eluting solvent: tetrahydrofuran

Flow rate: 1.0 mL/min

Sample concentration: 1.0% by mass

Sample injection amount: 100 μL

Detector: Differential Refractometer

Reference material: monodisperse polystyrene

The content of the resin (A) is preferably not less than 70% by mass, more preferably not less than 80% by mass, and further preferably not less than 85% by mass based on the total solid content of the radiation-sensitive resin composition.

(Other Resins)

The radiation-sensitive resin composition according to the present embodiment may contain, as another resin, a resin having higher content by mass of fluorine atoms than the above-described base resin (hereinafter, also referred to as a “high fluorine-containing resin). When the radiation-sensitive resin composition contains the high fluorine-containing resin, the high fluorine-containing resin can be localized in the surface layer of a resist film compared to the base resin, which as a result makes it possible to control the resist film so that the resist film can have a desired surface condition or a desired component distribution.

The high fluorine-containing resin preferably has, for example, the structural units (a1) and (a2) included in the base resin and a structural unit represented by the following formula (6) (hereinafter, also referred to as a “structural unit (a5)”).

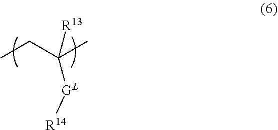

In the above formula (6), R¹³ is a hydrogen atom, a methyl group, or a trifluoromethyl group; G is a single bond, an oxygen atom, a sulfur atom, —COO—, —SO₂ONH—, —CONH—, or —OCONH—; R¹⁴ is a monovalent fluorinated chain hydrocarbon group having a carbon number of 1 to 20, or a monovalent fluorinated alicyclic hydrocarbon group having a carbon number of 3 to 20.

As R¹³ as described above, in terms of the copolymerizability of monomers resulting in the structural unit (a5), a hydrogen atom or a methyl group is preferred, and a methyl group is more preferred.

As G^{L} as described above, in terms of the copolymerizability of monomers resulting in the structural unit (a5), a single bond or —COO— is preferred, and —COO— is more preferred.

Example of the monovalent fluorinated chain hydrocarbon group having a carbon number of 1 to 20 represented by R¹⁴ as described above includes a group in which a part of or all of hydrogen atoms in the straight or branched chain alkyl group having a carbon number of 1 to 20 is/are substituted with a fluorine atom.

Example of the monovalent fluorinated alicyclic hydrocarbon group having a carbon number of 3 to 20 represented by R¹⁴ as described above includes a group in which a part of or all of hydrogen atoms in the monocyclic or polycyclic hydrocarbon group having a carbon number of 3 to 20 is/are substituted with a fluorine atom.

The R¹⁴ as described above is preferably a fluorinated chain hydrocarbon group, more preferably a fluorinated alkyl group, and further preferably 2,2,2-trifluoroethyl group, 1,1,1,3,3,3-hexafluoropropyl group and 5,5,5-trifluoro-1,1-diethylpentyl group.

When the high fluorine-containing resin has the structural unit (a5), the lower limit of the content of the structural unit (a5) is preferably 10 mol %, more preferably 15 mol %, even more preferably 20 mol %, particularly preferably 25 mol % with respect to the total amount of all the structural units constituting the high fluorine-containing resin. The upper limit of the content is preferably 60 mol %, more preferably 50 mol %, even more preferably 40 mol %. When the content of the structural unit (a5) is set to fall within the above range, the content by mass of fluorine atoms of the high fluorine-containing resin can more appropriately be adjusted to further promote the localization of the high fluorine-containing resin in the surface layer of a resist film.

The high fluorine-containing resin may have a fluorine atom-containing structural unit represented by the following formula (f-1) (hereinafter, also referred to as a “structural unit (a6)”) in addition to the structural unit (a5). When the high fluorine-containing resin has the structural unit (a6), solubility in an alkaline developing solution is improved, and therefore generation of development defects can be prevented.

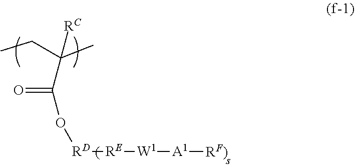

The structural unit (a6) is classified into two groups: a unit having an alkali soluble group (x); and a unit having a group (y) in which the solubility into the alkaline developing solution is increased by the dissociation by alkali (hereinafter, simply referred as an “alkali-dissociable group”). In both cases of (x) and (y), R^{C} in the above formula (f-1) is a hydrogen atom, a fluorine atom, a methyl group, or a trifluoromethyl group; R^{D} is a single bond, a hydrocarbon group having a carbon number of 1 to 20 with the valency of (s+1), a structure in which an oxygen atom, a sulfur atom, —NR^{dd}—, a carbonyl group, —COO— or —CONH— is connected to the terminal on R side of the hydrocarbon group, or a structure in which a part of hydrogen atoms in the hydrocarbon group is substituted with an organic group having a hetero atom; R^{dd} is a hydrogen atom, or a monovalent hydrocarbon group having a carbon number of 1 to 10; and s is an integer of 1 to 3.

When the structural unit (a6) has the alkali soluble group (x), R^{F} is a hydrogen atom; A¹ is an oxygen atom, —COO—* or —SO₂O—*; * refers to a bond to R^{F}; W¹ is a single bond, a hydrocarbon group having a carbon number of 1 to 20, or a divalent fluorinated hydrocarbon group. When A¹ is an oxygen atom, W¹ is a fluorinated hydrocarbon group having a fluorine atom or a fluoroalkyl group on the carbon atom connecting to A¹. R^{E} is a single bond, or a divalent organic group having a carbon number of 1 to 20. When s is 2 or 3, a plurality of R^{E}, W¹, A¹ and R^{F} may be each identical or different. The affinity of the high fluorine-containing resin into the alkaline developing solution can be improved by including the structural unit (a6) having the alkali soluble group (x), and thereby prevent from generating the development defect. As the structural unit (a6) having the alkali soluble group (x), particularly preferred is a structural unit in which A¹ is an oxygen atom and W¹ is a 1,1,1,3,3,3-hexafluoro-2,2-methanediyl group.

When the structural unit (a6) has the alkali-dissociable group (y), R^{F} is a monovalent organic group having carbon number of 1 to 30; A¹ is an oxygen atom, —NR^{aa}—, —COO—*, or —SO₂O—*; R^{aa} is a hydrogen atom, or a monovalent hydrocarbon group having a carbon number of 1 to 10; * refers to a bond to R^{F}; W¹ is a single bond, or a divalent fluorinated hydrocarbon group having a carbon number of 1 to 20; R^{E} is a single bond, or a divalent organic group having a carbon number of 1 to 20. When A¹ is —COO—* or —SO₂O—*, W¹ or R^{F} has a fluorine atom on the carbon atom connecting to A¹ or on the carbon atom adjacent to the carbon atom. When A¹ is an oxygen atom, W¹ and R^{E} are a single bond; R^{D} is a structure in which a carbonyl group is connected at the terminal on R^{E} side of the hydrocarbon group having a carbon number of 1 to 20; and R^{F} is an organic group having a fluorine atom. When s is 2 or 3, a plurality of R^{E}, W¹, A¹ and R^{F} may be each identical or different. The surface of the resist film is changed from hydrophobic to hydrophilic in the alkaline developing by including the structural unit (a6) having the alkali-dissociable group (y). As a result, the affinity of the high fluorine-containing resin into the alkaline developing solution can be significantly improved, and thereby prevent from generating the development defect more efficiently. As the structural unit (a6) having the alkali-dissociable group (y), particularly preferred is a structural unit in which A¹ is —COO—*, and R^{F} or W¹, or both is/are a fluorine atom.

In terms of the copolymerizability of monomers resulting in the structural unit (a6), R^{C} is preferably a hydrogen atom or a methyl group, and more preferably a methyl group.

When R^{E} is a divalent organic group, R^{E} is preferably a group having a lactone structure, more preferably a group having a polycyclic lactone structure, and further preferably a group having a norbornane lactone structure.

When the high fluorine-containing resin has the structural unit (a6), the lower limit of the content of the structural unit (a6) is preferably 10 mol %, more preferably 20 mol %, even more preferably 30 mol %, particularly preferably 35 mol % with respect to the total amount of all the structural units constituting the high fluorine-containing resin. The upper limit of the content is preferably 90 mol %, more preferably 75 mol %, even more preferably 60 mol %. When the content of the structural unit (a6) is set to fall within the above range, water repellency of a resist film during immersion exposure can further be improved.

The lower limit of Mw of the high fluorine-containing resin is preferably 1,000, more preferably 2,000, further preferably 3,000, and particularly preferably 5,000. The upper limit of Mw is preferably 50,000, more preferably 30,000, further preferably 20,000, and particularly preferably 15,000.

The lower limit of the Mw/Mn of the high fluorine-containing resin is typically 1, and more preferably 1.1. The upper limit of the Mw/Mn is typically 5, preferably 3, more preferably 2, and further preferably 1.7.

The lower limit of the content of the high fluorine-containing resin is preferably 0.1% by mass, more preferably 0.5% by mass, further preferably 1% by mass, and even further preferably 1.5% by mass based on the total solid content of the radiation-sensitive resin composition. The upper limit of the content is preferably 20% by mass, more preferably 15% by mass, further preferably 10% by mass, and particularly preferably 7% by mass.

The lower limit of the content of the high fluorine-containing resin is preferably 0.1 part by mass, more preferably 0.5 part by mass, further preferably 1 part by mass, and particularly preferably 1.5 part by mass based on 100 parts by mass of total base resins. The upper limit of the content is preferably 15 parts by mass, more preferably 10 parts by mass, further preferably 8 parts by mass, and particularly preferably 5 parts by mass.

By setting the content of the high fluorine-containing resin within the above range, the high fluorine-containing resin can be more effectively localized to the surface layer of the resist film, and as a result, the water repellency of the surface of the resist film during liquid immersion exposure can be further enhanced. The radiation-sensitive resin composition may contain one or two or more of high fluorine-containing resins.

(Method for Synthesizing High Fluorine-Containing Resin)

The high fluorine-containing resin can be synthesized by a method similar to the above-described method for synthesizing a base resin.

### (Compound (B))

The compound (B) can function as a quencher (photodegradable base) which captures an acid before exposure or in an unexposed portion. The compound (B) is represented by the following formula (1):

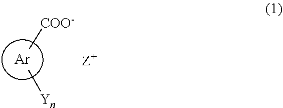

in the formula (1), Ar is a substituted or unsubstituted aromatic ring having 6 to 20 carbon atoms; n is an integer of 2 to 4; Z⁺ is a monovalent onium cation; a plurality of Ys are each independently a polar group; and at least one of the plurality of Ys is an —OH group or an —SH group bonded to a carbon atom adjacent to a carbon atom to which a COO⁻ group is bonded.

The radiation-sensitive resin composition contains the compound (B), which makes it possible to impart sensitivity, depth of focus, and process margins to the radiation-sensitive resin composition at high levels through the suppression of defects caused by variation in an exposure amount or a focus position due to improvement in the solubility of the resin in the alkaline developer.

In the formula (1), the substituted or unsubstituted aromatic ring having 6 to 20 carbon atoms is not particularly limited, and may have an aromatic heterocyclic structure in which a carbon atom forming a skeleton is substituted with a hetero atom, regardless of a monocyclic ring or a polycyclic ring, and a hydrogen atom on the carbon atom may be substituted with a substituent other than the polar group.

Examples of the aromatic ring include groups having a benzene ring structure, a naphthalene ring structure, a phenanthrene ring structure, and an anthracene ring structure and the like.

Examples of the hetero atom in the aromatic heterocyclic structure include an oxygen atom, a nitrogen atom, and a sulfur atom.

Examples of the aromatic heterocyclic structure include oxygen atom-containing heterocyclic structures such as a furan ring structure, a pyran ring structure, a benzofuran ring structure, and a benzopyran ring structure; nitrogen atom-containing heterocyclic structures such as a pyridine ring structure, a pyrimidine ring structure, and an indole ring structure; and sulfur atom-containing heterocyclic structures such as a thiophene ring structure.

Examples of the polar group include a hydroxy group, a sulfanyl group, a carboxy group, a cyano group, a nitro group, an amino group, a group having an ester bond, and a halogen atom.

Examples of the substituent include halogen atoms such as a fluorine atom, a chlorine atom, a bromine atom, and an iodine atom, an alkyl group, an alkoxy group, an alkoxycarbonyl group, an alkoxycarbonyloxy group, an acyl group, and an acyloxy group.

An example of the monovalent onium cation is a radioactive ray-degradable onium cation containing an element such as S, I, O, N, P, Cl, Br, F, As, Se, Sn, Sb, Te, or Bi. Examples of such a radioactive ray-degradable onium cation include a sulfonium cation, a tetrahydrothiophenium cation, a iodonium cation, a phosphonium cation, a diazonium cation, and a pyridinium cation. Among them, a sulfonium cation or a iodonium cation is preferred. The sulfonium cation or the iodonium cation is preferably represented by any of the following formulas (X-1) to (X-6).

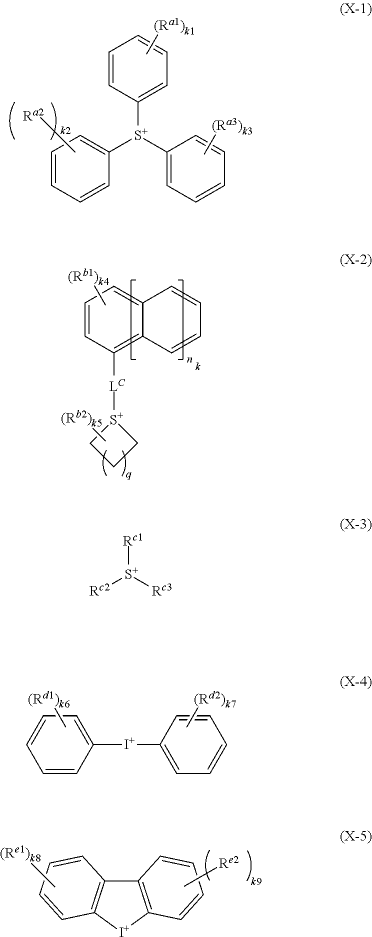

In the above formula (X-1), R^{a1}, R^{a2} and R^{a3} are each independently a substituted or unsubstituted, straight or branched chain alkyl group, alkoxy group or alkoxycarbonyloxy group having a carbon number of 1 to 12; a substituted or unsubstituted, monocyclic or polycyclic cycloalkyl group having a carbon number of 3 to 12; a substituted or unsubstituted aromatic hydrocarbon group having a carbon number of 6 to 12; a hydroxy group, —OSO₂—R^{P}, —SO₂—R^{Q} or —S—R^{T}; or a ring structure obtained by combining two or more of these groups. R^{P}, R^{Q} and R^{T} are each independently a substituted or unsubstituted, straight or branched chain alkyl group having a carbon number of 1 to 12; a substituted or unsubstituted alicyclic hydrocarbon group having a carbon number of 5 to 25; and a substituted or unsubstituted aromatic hydrocarbon group having a carbon number of 6 to 12. k1, k2 and k3 are each independently an integer of 0 to 5. When there are a plurality of R^{a1} to R^{a3} and a plurality of R^{P}, R^{Q} and R^{T}, a plurality of R^{a1} to R^{a3} and a plurality of R^{P}, R^{Q} and R^{T} may be each identical or different.

In the above formula (X-2), R^{b1} is a substituted or unsubstituted, straight chain or branched alkyl group or alkoxy group having a carbon number of 1 to 20; a substituted or unsubstituted acyl group having a carbon number of 2 to 8; or a substituted or unsubstituted aromatic hydrocarbon group having a carbon number of 6 to 8; or a hydroxy group. nₖ is 0 or 1. When nₖ is 0, k4 is an integer of 0 to 4. When nₖ is 1, k4 is an integer of 0 to 7. When there are a plurality of R^{b1}, a plurality of R^{b1} may be each identical or different. A plurality of R^{b1} may represent a ring structure obtained by combining them. R^{b2} is a substituted or unsubstituted, straight chain or branched alkyl group having a carbon number of 1 to 7; or a substituted or unsubstituted aromatic hydrocarbon group having a carbon number of 6 to 7. L^{C} is a single bond or divalent linking group. k5 is an integer of 0 to 4. When there are a plurality of R^{b2}, a plurality of R^{b2} may be each identical or different. A plurality of R^{b2} may represent a ring structure obtained by combining them. q is an integer of 0 to 3.

In the above formula (X-3), R^{c1}, R^{c2} and R^{c3} are each independently a substituted or unsubstituted, straight or branched chain alkyl group having a carbon number of 1 to 12 or a substituted or unsubstituted hydrocarbon group having a carbon number of 6 to 12.

In the above formula (X-4), R^{d1} and R^{d2} are each independently a substituted or unsubstituted, straight or branched chain alkyl group, alkoxy group or alkoxycarbonyl group having a carbon number of 1 to 12; a substituted or unsubstituted aromatic hydrocarbon group having a carbon number of 6 to 12; a halogen atom; a halogenated alkyl group having a carbon number of 1 to 4; a nitro group; or a ring structure obtained by combining two or more of these groups. k6 and k7 are each independently an integer of 0 to 5. When there are a plurality of R^{d1} and a plurality of R^{d2}, a plurality of R^{d1} and a plurality of R^{d2} may be each identical or different.

In the above formula (X-5), R^{e1} and R^{e2} are each independently a halogen atom; a substituted or unsubstituted straight or branched chain alkyl group having a carbon number of 1 to 12; or a substituted or unsubstituted aromatic hydrocarbon group having a carbon number of 6 to 12. k8 and k9 are each independently an integer of 0 to 4.

The compound (B) is preferably a compound represented by the following formula (1-1) (that is, a compound (b)):

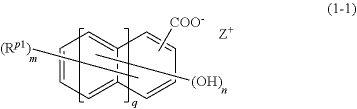

in the formula (1-1), R^{p1} is an alkoxy group, an alkoxycarbonyl group, a halogen atom, or an amino group; m is an integer of 0 to 3; when m is 2 or 3, a plurality of R^{p1}s are the same or different; n and Z⁺ have the same meanings as those in the above formula (1); q is an integer of 0 to 2; when q is 0, m+n is less than or equal to 5; and at least one OH group is bonded to the carbon atom adjacent to the carbon atom to which the COO⁻ group is bonded.

By adopting the compound (b) represented by the formula (1-1) as the compound (B), the polarity can be improved, whereby the sensitivity, the depth of focus, and the process margins can be efficiently improved.

In the formula (1-1), examples of the alkoxy group include a methoxy group, an ethoxy group, a propoxy group, and a butoxy group.

Examples of the alkoxycarbonyl group include a methoxycarbonyl group, an ethoxycarbonyl group, and a propoxycarbonyl group.

Examples of the halogen atom include a fluorine atom, a chlorine atom, a bromine atom, and an iodine atom.

m in the formula (1-1) is preferably 0 to 2, and more preferably 0 or 1. q is preferably 0 or 1. Furthermore, n is preferably 2 or 3.

Specific suitable examples of the compound represented by the formula (1-1) include the following formulae (1-1a) to (1-1i).

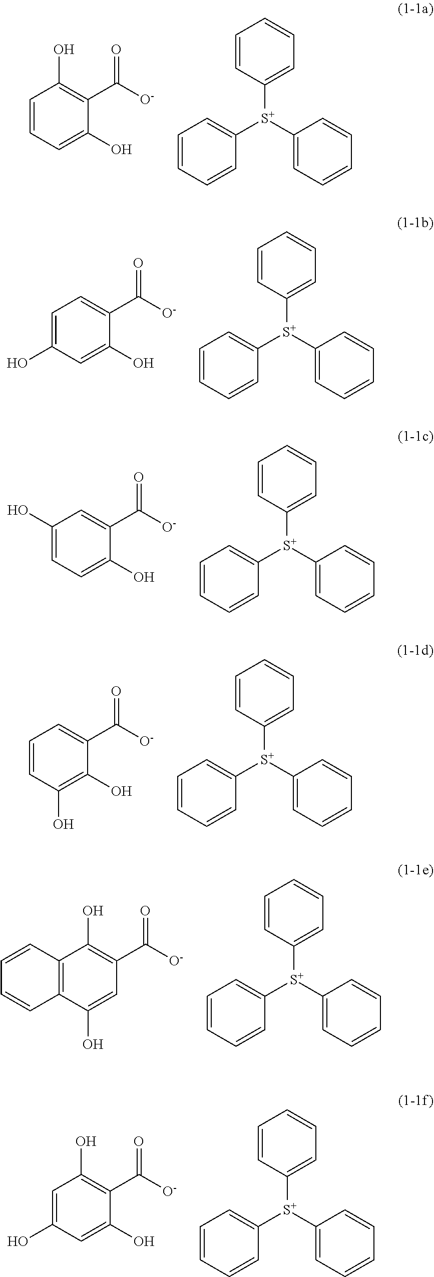

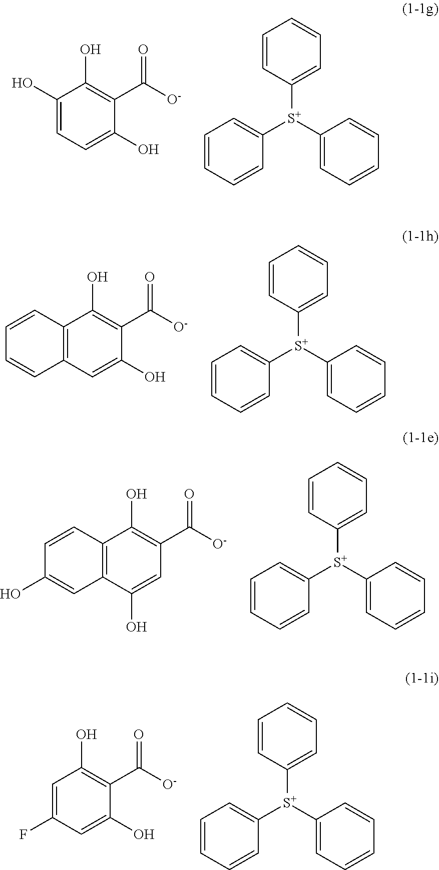

The content of the compound (B) is preferably 0.5 parts by mass or more and 100 parts by mass or less with respect to 100 parts by mass of the resin. The upper limit of the content is more preferably 50 parts by mass, and still more preferably 25 parts by mass. The lower limit of the content is more preferably 1 part by mass, and still more preferably 2 parts by mass. The content of the compound (B) is appropriately selected depending on the type of the resin (A) to be used, exposure conditions, required sensitivity, and the type and content of the radiation-sensitive acid generator (C) to be described later. Thereby, the solubility of the resin (A) can be obtained at adequate levels, and the sensitivity, the depth of focus, and the process margins can be exhibited at higher levels.

When the radiation-sensitive resin composition according to the present embodiment contains the radiation-sensitive acid generator to be described later, the upper limit of the molar ratio of the content of the compound (B) to the content of the radiation-sensitive acid generator is preferably 250 mol %, more preferably 200 mol %, still more preferably 100 mol %, and particularly preferably 50 mol %. Meanwhile, the lower limit of the molar ratio is preferably 3 mol-, more preferably 5 mol %, still more preferably 10 mol %, and particularly preferably 15 mol %.

### (Synthesis Method of Compound (B))

The compound (B) can be typically synthesized by reacting a benzoic acid derivative corresponding to an anion moiety with sulfonium chloride corresponding to a cation moiety under basic conditions to promote salt exchange. Similarly, the compound (B) having another structure can be synthesized by appropriately selecting precursors corresponding to the anion moiety and the cation moiety.

### (Radiation-Sensitive Acid Generator (C))

The radiation-sensitive acid generator (C) is a component which generates an acid by exposure. When the resin includes the structural unit (a2) having an acid-dissociable group, the acid generated by exposure can dissociate the acid-dissociable group of the structural unit (a2) to generate a carboxy group or the like. This function is different from the function of the compound (B) which suppresses the diffusion of the acid generated from the radiation-sensitive acid generator (C) in the unexposed portion without substantially dissociating the acid-dissociable group or the like of the structural unit (a2) of the resin or the like under a pattern formation condition using the radiation-sensitive resin composition. The acid generated from the radiation-sensitive acid generator (C) can be said to be a relatively stronger acid (an acid having a smaller pKa) than the acid generated from the compound (B). The functions of the compound (B) and radiation-sensitive acid generator (C) are determined by energy required for dissociating the acid-dissociable group of the structural unit (a2) of the resin or the like, and a thermal energy condition given when a pattern is formed using the radiation-sensitive resin composition, and the like. The contained form of the radiation-sensitive acid generator in the radiation-sensitive resin composition may be a form in which the radiation-sensitive acid generator is present alone as a compound (released from a polymer), a form in which the radiation-sensitive acid generator is incorporated as a part of a polymer, or both of these forms, but a form in which the radiation-sensitive acid generator is present alone as a compound is preferable.

When the radiation-sensitive resin composition contains the radiation-sensitive acid generator (C), the polarity of the resin in the exposed portion increases, whereby the resin in the exposed portion is soluble in the developer in the case of alkaline aqueous solution development, and is poorly soluble in the developer in the case of organic solvent development.

Examples of the radiation-sensitive acid generator (C) include an onium salt compound, a sulfonimide compound, a halogen-containing compound, and a diazoketone compound. Examples of the onium salt compound include a sulfonium salt, a tetrahydrothiophenium salt, an iodonium salt, a phosphonium salt, a diazonium salt, and a pyridinium salt. Among them, a sulfonium salt and an iodonium salt are preferable.

Examples of the acid generated by exposure include an acid which generates sulfonic acid by exposure. Examples of such an acid include a compound in which one or more fluorine atoms or fluorinated hydrocarbon groups substitute a carbon atom adjacent to a sulfo group. Among them, the radiation-sensitive acid generator (C) is particularly preferably one having a cyclic structure.

These radiation-sensitive acid generators may be used alone or in combination of two or more thereof. The content of the radiation-sensitive acid generator may be 5 parts by mass or more with respect to 100 parts by mass of the resin from the viewpoint of securing sensitivity and developability as a resist, but is preferably 10 parts by mass or more from the viewpoint of sensitivity, depth of focus, and process margins. The lower limit of the content is more preferably 12 parts by mass, and still more preferably 15 parts by mass. The upper limit of the content is preferably 60 parts by mass, more preferably 50 parts by mass, and still more preferably 40 parts by mass.

### (Solvent (D))

The radiation-sensitive resin composition according to the present embodiment contains a solvent (D). The solvent (D) is not particularly limited as long as the solvent (D) is a solvent capable of dissolving or dispersing at least the resin (A) and the compound (B), and the radiation-sensitive acid generator (C) and the like contained as desired.

Examples of the solvent (D) include an alcohol-based solvent, an ether-based solvent, a ketone-based solvent, an amide-based solvent, an ester-based solvent, and a hydrocarbon-based solvent.

Examples of the alcohol-based solvent include:

a monoalcohol-based solvent having a carbon number of 1 to 18, including iso-propanol, 4-methyl-2-pentanol, 3-methoxybutanol, n-hexanol, 2-ethylhexanol, furfuryl alcohol, cyclohexanol, 3,3,5-trimethylcyclohexanol, and diacetone alcohol;

a polyhydric alcohol having a carbon number of 2 to 18, including ethylene glycol, 1,2-propylene glycol, 2-methyl-2,4-pentanediol, 2,5-hexanediol, diethylene glycol, dipropylene glycol, triethylene glycol, and tripropylene glycol; and

a partially etherized polyhydric alcohol-based solvent in which a part of hydroxy groups in the polyhydric alcohol-based solvent is etherized.

Examples of the ether-based solvent include:

a dialkyl ether-based solvent, including diethyl ether, dipropyl ether, and dibutyl ether;

a cyclic ether-based solvent, including tetrahydrofuran and tetrahydropyran;

an ether-based solvent having an aromatic ring, including diphenylether and anisole (methyl phenyl ether); and

an etherized polyhydric alcohol-based solvent in which a hydroxy group in the polyhydric alcohol-based solvent is etherized.

Examples of the ketone-based solvent include:

a chain ketone-based solvent, including acetone, butanone, and methyl-iso-butyl ketone;

a cyclic ketone-based solvent, including cyclopentanone, cyclohexanone, and methylcyclohexanone; and

2,4-pentanedione, acetonylacetone, and acetophenone.

Examples of the amide-based solvent include:

a cyclic amide-based solvent, including N,N′-dimethyl imidazolidinone and N-methylpyrrolidone; and

a chain amide-based solvent, including N-methylformamide, N,N-dimethylformamide, N,N-diethylformamide, acetamide, N-methylacetamide, N,N-dimethylacetamide, and N-methylpropionamide.

Examples of the ester-based solvent include:

a monocarboxylate ester-based solvent, including n-butyl acetate and ethyl lactate;

a partially etherized polyhydric alcohol acetate-based solvent, including diethylene glycol mono-n-butyl ether acetate, propylene glycol monomethyl ether acetate, and dipropylene glycol monomethyl ether acetate;

a lactone-based solvent, including γ-butyrolactone and valerolactone;

a carbonate-based solvent, including diethyl carbonate, ethylene carbonate, and propylene carbonate; and

a polyhydric carboxylic acid diester-based solvent, including propylene glycol diacetate, methoxy triglycol acetate, diethyl oxalate, ethyl acetoacetate, ethyl lactate, and diethyl phthalate.

Examples of the hydrocarbon-based solvent include:

an aliphatic hydrocarbon-based solvent, including n-hexane, cyclohexane, and methylcyclohexane;

an aromatic hydrocarbon-based solvent, including benzene, toluene, di-iso-propylbenzene, and n-amylnaphthalene.

Among them, the ester-based solvent or the ketone-based solvent is preferred. The partially etherized polyhydric alcohol acetate-based solvent, the cyclic ketone-based solvent, or the lactone-based solvent is more preferred. Propylene glycol monomethyl ether acetate, cyclohexanone, or γ-butyrolactone is still more preferred. The radiation-sensitive resin composition may include one type of the solvent, or two or more types of the solvents in combination.

<Another Optional Component>

The radiation-sensitive resin composition may contain another optional component other than the above-descried components. Examples of the another optional component include a cross-linking agent, a localization enhancing agent, a surfactant, an alicyclic backbone-containing compound, and a sensitizer. These other optional components may be used singly or in combination of two or more of them.

(Cross-Linking Agent)

The cross-linking agent is a compound having two or more functional groups. The cross-linking agent causes a cross-linking reaction in a polymer component (1) by an acid catalytic reaction in a baking after a one-shot exposing to increase the molecular weight of the polymer component (1) so that the solubility of a pattern-exposed part in a developer is reduced. Examples of the functional group include a (meth)acryloyl group, a hydroxymethyl group, an alkoxymethyl group, an epoxy group, and a vinyl ether group.

(Localization Enhancing Agent)

The localization enhancing agent has an effect of localizing the high fluorine-containing resin on the surface of the resist film more effectively. The added amount of the high fluorine-containing resin can be decreased compared to the traditionally added amount by including the localization enhancing agent in the radiation-sensitive resin composition. The localization enhancing agent can further prevent from eluting the ingredient of the composition from the resist film to an immersion medium and carry out the immersion exposure at higher speed with a high-speed scan, while maintaining the lithography properties of the radiation-sensitive resin composition. As a result, the hydrophobicity of the surface of the resist film can be improved, resulting in the prevention of the defect due to the immersion, for example, the watermark defect. Example of the compound which may be used as the localization enhancing agent includes a low molecular weight compound having a specific dielectric constant of not less than 30 and not more than 200 and a boiling point of 100° C. or more at 1 atm. Specific examples of the compound include a lactone compound, a carbonate compound, a nitrile compound, and a polyhydric alcohol.

Examples of the lactone compound include γ-butyrolactone, valerolactone, mevalonic lactone, and norbornane lactone.

Examples of the carbonate compound include propylene carbonate, ethylene carbonate, butylene carbonate, and vinylene carbonate.

Examples of the nitrile compound include succinonitrile.

Examples of the polyhydric alcohol include glycerin.

The lower limit of the content of the localization enhancing agent is preferably 10 parts by mass, more preferably 15 parts by mass, still more preferably 20 parts by mass, and still more preferably 25 parts by mass, with respect to 100 parts by mass of the total amount of the resin in the radiation-sensitive resin composition. The upper limit of the content is preferably 300 parts by mass, more preferably 200 parts by mass, still more preferably 100 parts by mass, and particularly preferably 80 parts by mass. The radiation-sensitive resin composition may contain one or two or more of localization enhancing agents.

(Surfactant)

The surfactant has an effect of improving the coating properties, the striation, and the developability of the composition. Examples of the surfactant include a nonionic surfactant, including polyoxyethylene lauryl ether, polyoxyethylene stearyl ether, polyoxyethylene oleyl ether, polyoxyethylene n-octylphenyl ether, polyoxyethylene n-nonylphenyl ether, polyethylene glycol dilaurate, and polyethylene glycol distearate. Examples of the surfactant which is commercially available include KP341 (manufactured by Shin-Etsu Chemical Co., Ltd.), POLYFLOW No. 75, POLYFLOW No. 95 (all manufactured by Kyoeisha Chemical Co., Ltd.), EFTOP EF301, EFTOP EF303, EFTOP EF352 (all manufactured by Tokem Products), Megafac F171, Megafac F173 (all manufactured by DIC), Fluorad FC430, Fluorad FC431 (all manufactured by Sumitomo 3M Limited.), AsahiGuard AG710, Surflon S-382, Surflon SC-101, Surflon SC-102, Surflon SC-103, Surflon SC-104, Surflon SC-105, Surflon SC-106 (all manufactured by Asahi Glass Co., Ltd.). The content of the surfactant in the radiation-sensitive resin composition is typically not more than 2 parts by mass based on 100 parts by mass of total resins.

(Alicyclic Backbone-Containing Compound)

The alicyclic backbone-containing compound has an effect of improving the dry etching resistance, the shape of the pattern, the adhesiveness between the substrate, and the like.

Examples of the alicyclic backbone-containing compound include:

adamantane derivatives, including 1-adamantane carboxylic acid, 2-adamantanone, and t-butyl 1-adamantane carboxylate;

deoxycholic acid esters, including t-butyl deoxycholate, t-butoxycarbonylmethyl deoxycholate, and 2-ethoxyethyl deoxycholate;

lithocholic acid esters, including t-butyl lithocholate, t-butoxycarbonylmethyl lithocholate, and 2-ethoxyethyl lithocholate; and

3-[2-hydroxy-2,2-bis(trifluoromethyl)ethyl]tetracyclo[4.4.0.1(2,5).1(7,10)]dode cane, and 2-hydroxy-9-methoxycarbonyl-5-oxo-4-oxa-tricyclo[4.2.1.0(3,7)]nonane. The content of the alicyclic backbone-containing compound in the radiation-sensitive resin composition is typically not more than 5 parts by mass based on 100 parts by mass of total resins.

(Sensitizer)

The sensitizer has the function of increasing the amount of an acid generated from the radiation-sensitive acid generator or the like, and is effective at improving “apparent sensitivity” of the radiation-sensitive resin composition.

Examples of the sensitizer include carbazoles, acetophenones, benzophenones, naphthalenes, phenols, biacetyl, eosin, rose bengal, pyrenes, anthracenes, and phenothiazines. These sensitizers may be used singly or in combination of two or more of them. The content of the sensitizer in the radiation-sensitive resin composition is usually 2 parts by mass or less per 100 parts by mass of the resin.

<Method for Preparing Radiation-Sensitive Resin Composition>

The radiation-sensitive resin composition can be prepared by, for example, mixing the resin (A), the compound (B), the radiation-sensitive acid generator (C), the high fluorine-containing resin, if necessary, and the solvent (D) in a predetermined ratio. The radiation-sensitive resin composition is preferably filtered through, for example, a filter having a pore diameter of about 0.05 μm after mixing. The solid matter concentration of the radiation-sensitive resin composition is usually 0.1 mass % to 50 mass %, preferably 0.5 mass % to 30 mass %, more preferably 1 mass % to 20 mass %.

<<Method for Forming Resist Pattern>>

A method for forming a resist pattern according to the present embodiment includes:

(1) applying the radiation-sensitive resin composition directly or indirectly onto a substrate to form a resist film (hereinafter, also referred to as a “resist film-forming”);

(2) exposing the resist film (hereinafter, also referred to as an “exposing”); and

(3) developing the exposed resist film (hereinafter, also referred to as a “developing”).

The method for forming a resist pattern uses the above-described radiation-sensitive resin composition excellent in sensitivity in the exposing, depth of focus and process margins, and therefore a high-quality resist pattern can be formed. Hereinbelow, each of the resist film-forming, exposing and developing will be described.

[Resist Film Forming]

In the resist film-forming (1), a resist film is formed with the radiation-sensitive resin composition. Examples of the substrate on which the resist film is formed include one traditionally known in the art, including a silicon wafer, silicon dioxide, and a wafer coated with aluminum. An organic or inorganic antireflection film may be formed on the substrate, as disclosed in JP-B-06-12452 and JP-A-59-93448. Examples of the applicating method include a rotary coating (spin coating), flow casting, and roll coating. After applicating, a prebake (PB) may be carried out in order to evaporate the solvent in the film, if needed. The temperature of PB is typically from 60° C. to 140° C., and preferably from 80° C. to 120° C. The duration of PB is typically from 5 seconds to 600 seconds, and preferably from 10 seconds to 300 seconds. The thickness of the resist film formed is preferably from 10 nm to 1,000 nm, and more preferably from 10 nm to 500 nm.

When the immersion exposure is carried out, irrespective of presence of a water repellent polymer additive such as the high fluorine-containing resin in the radiation-sensitive resin composition, the formed resist film may have a protective film for the immersion which is not soluble into the immersion liquid on the film in order to prevent a direct contact between the immersion liquid and the resist film. As the protective film for the immersion, a solvent-removable protective film that is removed with a solvent before the developing (for example, see JP-A-2006-227632); or a developer-removable protective film that is removed during the development of the developing (for example, see WO2005-069076 and WO2006-035790) may be used. In terms of the throughput, the developer-removable protective film is preferably used.

Further, when the next exposing is performed using a radioactive ray having a wavelength of 50 nm or less, a resin having the structural units (a1) and (a2) is preferably used as the base resin in the composition.

[Exposing]

In the exposing (2), the resist film formed in the resist film-forming (1) is exposed by irradiating with a radioactive ray through a photomask (optionally through an immersion medium such as water). Examples of the radioactive ray used for the exposure include visible ray, ultraviolet ray, far ultraviolet ray, extreme ultraviolet ray (EUV); an electromagnetic wave including X ray and y ray; an electron beam; and a charged particle radiation such as a ray. Among them, far ultraviolet ray, an electron beam, or EUV is preferred. ArF excimer laser light (wavelength is 193 nm), KrF excimer laser light (wavelength is 248 nm), an electron beam, or EUV is more preferred. An electron beam or EUV having a wavelength of 50 nm or less which is identified as the next generation exposing technology is further preferred.

When the exposure is carried out by immersion exposure, examples of the immersion liquid include water and fluorine-based inert liquid. The immersion liquid is preferably a liquid which is transparent with respect to the exposing wavelength, and has a minimum temperature factor of the refractive index so that the distortion of the light image reflected on the film becomes minimum. However, when the exposing light source is ArF excimer laser light (wavelength is 193 nm), water is preferably used because of the ease of availability and ease of handling in addition to the above considerations. When water is used, a small proportion of an additive that decreases the surface tension of water and increases the surface activity may be added. Preferably, the additive cannot dissolve the resist film on the wafer and can neglect an influence on an optical coating at an under surface of a lens. The water used is preferably distilled water.

After the exposure, post exposure bake (PEB) is preferably carried out to promote the dissociation of the acid-dissociable group in the resin by the acid generated from the radiation-sensitive acid generator with the exposure in the exposed part of the resist film. The difference of solubility into the developer between the exposed part and the non-exposed part is generated by the PEB. The temperature of PEB is typically from 50° C. to 180° C., and preferably from 80° C. to 130° C. The duration of PEB is typically from 5 seconds to 600 seconds, and preferably from 10 seconds to 300 seconds.

[Developing]

In the developing (3), the resist film exposed in the exposing (2) is developed. By this, the predetermined resist pattern can be formed. After the development, the resist pattern is washed with a rinse solution such as water or alcohol, and the dried, in general.

Examples of the developer used for the development include, in the alkaline development, an alkaline aqueous solution obtained by dissolving at least one alkaline compound such as sodium hydroxide, potassium hydroxide, sodium carbonate, sodium silicate, sodium metasilicate, ammonia water, ethylamine, n-propylamine, diethylamine, di-n-propylamine, triethylamine, methyldiethylamine, ethyldimethylamine, triethanolamine, tetramethyl ammonium hydroxide (TMAH), pyrrole, piperidine, choline, 1,8-diazabicyclo-[5.4.0]-7-undecene, 1,5-diazabicyclo-[4.3.0]-5-nonene. Among them, an aqueous TMAH solution is preferred, and 2.38% by mass of aqueous TMAH solution is more preferred.

In the case of the development with organic solvent, examples of the solvent include an organic solvent, including a hydrocarbon-based solvent, an ether-based solvent, an ester-based solvent, a ketone-based solvent, and an alcohol-based solvent; and a solvent containing an organic solvent. Examples of the organic solvent include one, two or more solvents listed as the solvent for the radiation-sensitive resin composition. Among them, an ester-based solvent or a ketone-based solvent is preferred. The ester-based solvent is preferably an acetate ester-based solvent, and more preferably n-butyl acetate or amyl acetate. The ketone-based solvent is preferably a chain ketone, and more preferably 2-heptanone. The content of the organic solvent in the developer is preferably not less than 80% by mass, more preferably not less than 90% by mass, further preferably not less than 95% by mass, and particularly preferably not less than 99% by mass. Examples of the ingredient other than the organic solvent in the developer include water and silicone oil.

Examples of the developing method include a method of dipping the substrate in a tank filled with the developer for a given time (dip method); a method of developing by putting and leaving the developer on the surface of the substrate with the surface tension for a given time (paddle method); a method of spraying the developer on the surface of the substrate (spray method); and a method of injecting the developer while scanning an injection nozzle for the developer at a constant rate on the substrate rolling at a constant rate (dynamic dispense method).

### Another Embodiment

Hereinafter, another embodiment will be described focusing on points different from those of the first embodiment. Examples of another embodiment include a radiation-sensitive resin composition containing: a resin including the structural unit (a2) and not containing a structural unit having a phenolic hydroxyl group; the compound (B); and the radiation-sensitive acid generator (C), the radiation-sensitive resin composition having a content of the radiation-sensitive acid generator (C) of 10 parts by mass or more with respect to 100 parts by mass of the resin, and a resist pattern forming method using the radiation-sensitive resin composition and ArF excimer laser light. In this embodiment, the resin is preferably a resin including the structural unit (a2) and at least one structural unit selected from the group consisting of the structural unit (a3) and the structural unit (a4). The content ratios of these structural units may be proportional distributed to each of the structural units based on the content ratios in the resin (A), with a content obtained by excluding the structural unit (a1) from the resin (A) as 100 mol %. The suitable lower limit and upper limit of the content of the radiation-sensitive acid generator (C) are the same as those of the first embodiment except that the content of the radiation-sensitive acid generator (C) is 10 parts by mass or more with respect to 100 parts by mass of the resin. Preferable embodiments of the types and contents of the compound (B), solvent (D), and other optional components are the same as those in the first embodiment. Regarding the method for forming a resist pattern using the radiation-sensitive resin composition, preferable embodiments of resist film-forming (1), exposing (2) and developing (3) are the same as those of the first embodiment except that ArF excimer laser light is used in the exposing (2).

### EXAMPLES

Hereinafter, the present invention will be specifically described with reference to Examples, but the present invention is not limited to these Examples. Physical property values in Examples were measured as follows.

### [Weight-Average Molecular Weight (Mw) and Number-Average Molecular Weight (Mn)]

The Mw and Mn of each polymer were measured by using GPC columns (two G2000 HXL columns, one G3000 HXL column, and one G4000 HXL column) manufactured by Tosoh Corp., and using gel permeation chromatography (GPC) with a mono-dispersed polystyrene as a standard under the following analysis conditions: flow rate: 1.0 mL/min, elution solvent: tetrahydrofuran, sample concentration: 1.0% by mass, sample injected volume: 100 μL, column temperature: 40° C., and detector: differential refractometer. Dispersity (Mw/Mn) was calculated from results of the measured Mw and Mn.

### <Synthesis of Resin (A)>

Monomers used for synthesis of resins (A) in Examples, Comparative Examples, and Reference Examples are shown below.

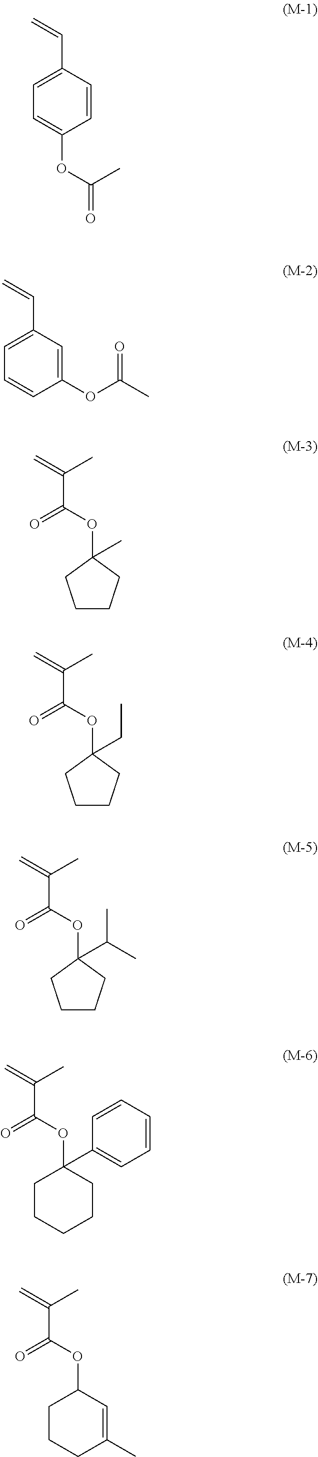

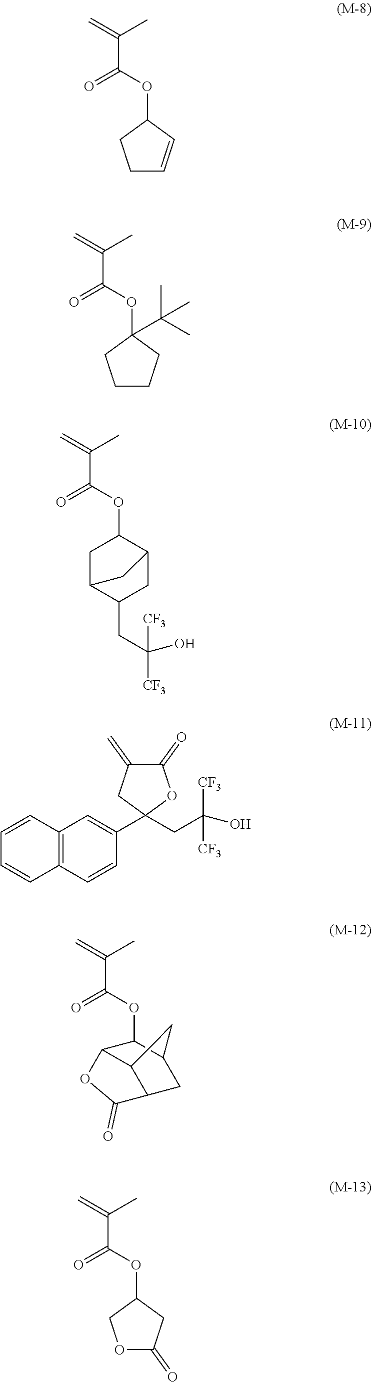 [SMILES: C=C(C)C(=O)OC1C=CCC1 ; C=C(C)C(=O)OC1(C(C)(C)C)CCCC1 ; C=C(C)C(=O)OC1CC2CC1CC2CC(O)(C(F)(F)F)C(F)(F)F ; C=C1CC(CC(O)(C(F)(F)F)C(F)(F)F)(c2ccc3ccccc3c2)OC1=O ; C=C(C)C(=O)OC1C2CC3C(=O)OC1C3C2 ; C=C(C)C(=O)OC1COC(=O)C1]

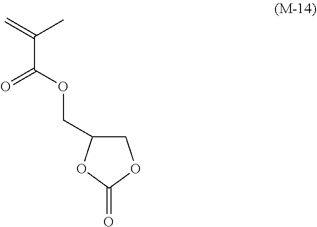

### [Synthesis Example 1] Synthesis of Resin (A-1)

A compound (M-1) and a compound (M-3) were dissolved in 1-methoxy-2-propanol (200 parts by mass based on the total amount of monomers) at a molar ratio of 40/60. Next, 6 mol % of azobisisobutyronitrile as an initiator was added into the total of the monomers to prepare a monomer solution. Meanwhile, 1-methoxy-2-propanol (100 parts by mass with respect to the total amount of monomers) was added into an empty reaction vessel, and heated to 85° C. while being stirred. Next, the monomer solution prepared above was added dropwise thereto over 3 hours, and then the mixture was further heated at 85° C. for 3 hours to perform a polymerization reaction for a total of 6 hours. After the completion of the polymerization reaction, the polymerization solution was cooled to room temperature.

The cooled polymerization solution was charged into hexane (500 parts by mass with respect to the polymerization solution), and a precipitated white powder was separated by filtration. The white powder separated by filtration was washed twice with 100 parts by mass of hexane relative to the polymerization solution, then separated by filtration, and dissolved in 1-methoxy-2-propanol (300 parts by mass). Next, methanol (500 parts by mass), triethylamine (50 parts by mass), and ultrapure water (10 parts by mass) were added to the resulting solution, and a hydrolysis reaction was performed at 70° C. for 6 hours with stirring.

After the completion of the reaction, the remaining solvent was distilled off, and the obtained solid was dissolved in acetone (100 parts by mass). The resulting solution was added dropwise into 500 parts by mass of water to permit the coagulation of the resin. The obtained solid was separated by filtration. The solid was dried at 50° C. for 12 hours to synthesize a white powdery resin (A-1).

### Synthesis Examples 2 to 9

Resins (A-2) to (A-9) were also synthesized in the same manner as in Synthesis Example 1 except that monomer species and ratios were changed to compositions shown in Table 1.

TABLE 1

| | | Monomer 1 | | Monomer 2 | | Monomer 3 | | | |
|---|---|---|---|---|---|---|---|---|---|
| | | | Used | | Used | | Used | | |
| | | | amount | | amount | | amount | | |
| | Resin (A) | Type | (mol %) | Type | (mol %) | Type | (mol %) | Mw | Mw/Mn |
| Synthesis | A-1 | M-1 | 40 | M-3 | 60 | — | — | 5700 | 1,61 |
| Examples | A-2 | M-1 | 40 | M-4 | 60 | — | — | 5800 | 1.64 |
| | A-3 | M-1 | 30 | M-5 | 60 | M-2 | 10 | 6100 | 1.65 |
| | A-4 | M-1 | 40 | M-6 | 60 | — | — | 6200 | 1.50 |
| | A-5 | M-1 | 40 | M-7 | 60 | — | — | 5500 | 1.54 |
| | A-6 | M-1 | 40 | M-8 | 60 | — | — | 5400 | 1.53 |
| | A-7 | M-1 | 40 | M-9 | 60 | — | — | 6000 | 1.67 |
| | A-8 | M-1 | 30 | M-3 | 60 | M-10 | 10 | 6900 | 1.70 |
| | A-9 | M-1 | 30 | M-3 | 60 | M-11 | 10 | 6800 | 1.65 |

*M-1 and M-2 have a hydroxybenzene structure after undergoing polymerization and hydrolysis.

### Synthesis Example 10

### (Synthesis of Resin (A-10))

A monomer (M-3) and a monomer (M-12) were dissolved in 2-butanone (200 parts by mass with respect to the total amount of monomers) at a molar ratio of 60/40, and AIBN (azobisisobutyronitrile) (3 mol % with respect to 100 mol % in total of all monomers used) as an initiator was added thereto to prepare a monomer solution. 2-butanone (100 parts by mass with respect to the total amount of monomers) was placed in an empty reaction vessel, and purged with nitrogen for 30 minutes. Then, a temperature in the reaction vessel was set to 80° C., and the monomer solution was added dropwise thereto over 3 hours while being stirred. The polymerization reaction was performed for 6 hours with the start of the dropwise addition as the start time of the polymerization reaction. After the completion of the polymerization reaction, the polymerization solution was water-cooled to 30° C. or lower. The cooled polymerization solution was charged into methanol (2,000 parts by mass), and a precipitated white powder was separated by filtration. The white powder separated by filtration was washed with methanol twice, then separated by filtration, and dried at 50° C. for 24 hours to obtain a white powdery polymer (A-10) (yield: 80%). The Mw of the polymer (A-10) was 7,800, and Mw/Mn was 1.51. As a result of ¹³C-NMR analysis, the content ratios of the structural units derived from (M-3) and (M-12) were respectively 58.9 mol % and 41.1 mol %.

### Synthesis Examples 11 and 12

Resins (A-11) and (A-12) were also synthesized in the same manner as in the above Synthesis Example 10 except that monomer species and ratios were changed to compositions shown in Table 2.

TABLE 2

| | | Monomer 1 | | Monomer 2 | | Monomer 3 | | | |
|---|---|---|---|---|---|---|---|---|---|
| | | | Used | | Used | | Used | | |
| | | | amount | | amount | | amount | | |
| | Resin (A) | Type | (mol %) | Type | (mol %) | Type | (mol %) | Mw | Mw/Mn |
| Synthesis | A-10 | M-3 | 60 | M-12 | 40 | — | — | 7800 | 1,51 |
| Examples | A-11 | M-3 | 60 | M-13 | 40 | — | — | 7700 | 1.65 |
| | A-12 | M-3 | 60 | M-14 | 40 | — | — | 6900 | 1.54 |

### <Synthesis of Compound (B)>

### (Synthesis of Compound (B-1))

A compound (B-1) was synthesized according to the following reaction scheme.

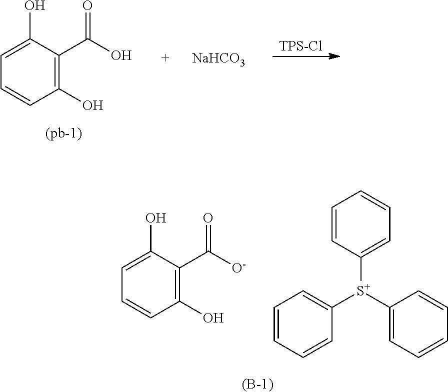

97.4 mmol of sodium hydrogen carbonate and 200 g of water were added into a reaction vessel. After the confirmation of the dissolution, 64.9 mmol of 2,6-dihydroxybenzoic acid was added thereto. The mixture was stirred at room temperature for 1 hour, and 300 g of dichloromethane and 64.9 mmol of triphenylsulfonium chloride were then added thereto. The mixture was stirred at room temperature for 2 hours, and an organic layer was then separated. The obtained organic layer was washed with water. The organic layer was dried over sodium sulfate, and the solvent was then distilled off, followed by recrystallizing to obtain a target compound (B-1).

### (Synthesis of Compounds (B-2) to (B-9))

Onium salt compounds represented by the following formulae (B-2) to (B-6) were synthesized in the same manner as in Example 1 by appropriately selecting precursors.

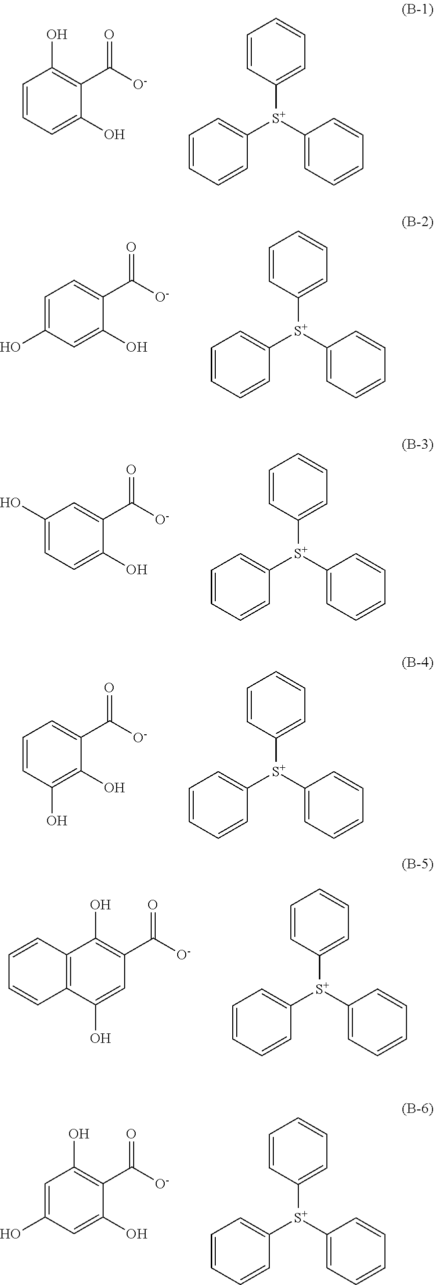

A compound represented by the following formula (CB-1) was used as an acid diffusion controlling agent in Comparative Examples.

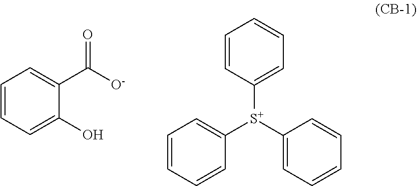 [SMILES: O=C([O-])c1ccccc1O ; c1ccc([S+](c2ccccc2)c2ccccc2)cc1]

<Radiation-Sensitive Acid Generator (C)>

As the radiation-sensitive acid generator (C), compounds represented by the following formulae (C-1) to (C-6) were used.

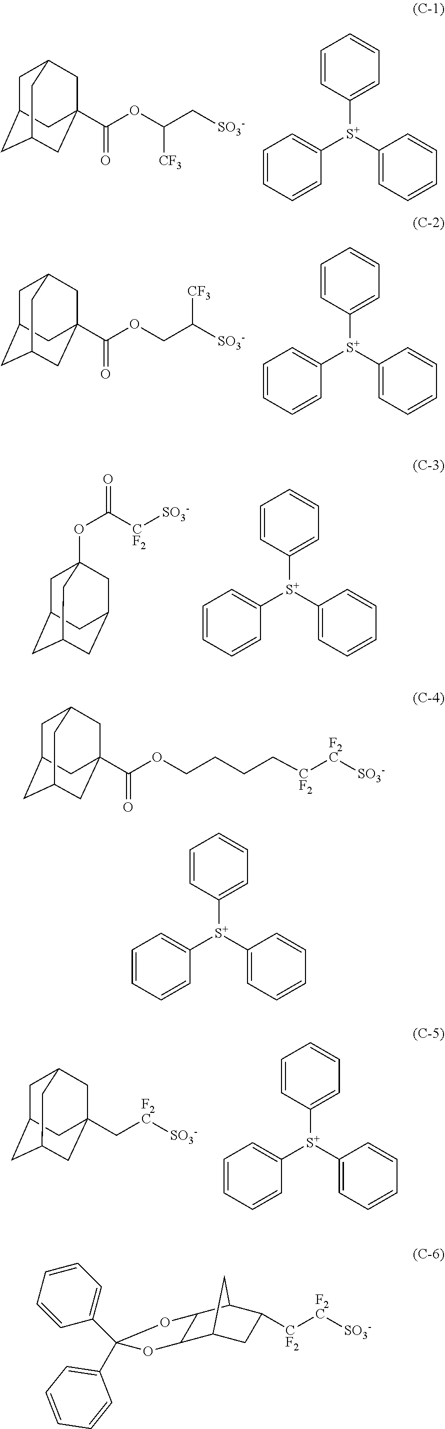

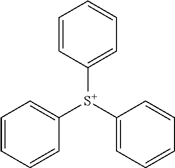 [SMILES: c1ccc([S+](c2ccccc2)c2ccccc2)cc1]

### <Solvent (D)>

The following solvents were used as the solvent (D).

D-1: propylene glycol monomethyl ether acetate

D-2: propylene glycol 1-monomethyl ether

D-3: cyclohexanone

D-4: γ-butyrolactone

### Example 1

A radiation-sensitive resin composition (R-1) was prepared by blending 100 parts by mass of (A-1) as the resin, 20 parts by mass of (C-1) as the radiation-sensitive acid generator, 20 mol % of the compound (B-1) as the acid diffusion controlling agent with respect to (C-1), 4,800 parts by mass of (D-1) and 2,000 parts by mass of (D-2) as the solvent (D).

### Examples 2 to 21 and Comparative Example 1

Radiation-sensitive resin compositions (R-2) to (R-21) and (CR-1) were prepared in the same manner as in Example 1 except that components of types and blending amounts shown in the following Table 3 were used.

TABLE 3

| | Radiation- | | | Compound (B) | | Radiation-sensitive | | | |
|---|---|---|---|---|---|---|---|---|---|
| | sensitive | Resin (A) | | | with respect | acid generator (C) | | Solvent (D) | |
| | resin | | Parts by | | to (C) | | Parts by | | Parts by |
| | combosition | Type | mass | Type | mol % | Type | mass | Solvent (D) | mass |
| Example 1 | R-1 | A-1 | 100 | B-1 | 20 | C-1 | 20 | D-1/D-2 | 4800/2000 |
| Example 2 | R-2 | A-1 | 100 | B-2 | 20 | C-1 | 20 | D-1/D-2 | 4800/2000 |
| Example 3 | R-3 | A-1 | 100 | B-3 | 20 | C-1 | 20 | D-1/D-2 | 4800/2000 |
| Example 4 | R-4 | A-1 | 100 | B-4 | 20 | C-1 | 20 | D-1/D-2 | 4800/2000 |
| Example 5 | R-5 | A-1 | 100 | B-5 | 20 | C-1 | 20 | D-1/D-2 | 4800/7000 |
| Example 6 | R-6 | A-1 | 100 | B-6 | 20 | C-1 | 20 | D-1/D-2 | 4800/7000 |
| Example 7 | R-7 | A-1 | 100 | B-1 | 20 | C-2 | 20 | D-1/D-2 | 4600/2000 |
| Example 6 | R-8 | A-1 | 100 | B-1 | 20 | C-3 | 20 | D-1/D-2 | 4600/2000 |
| Example 9 | R-9 | A-1 | 100 | B-1 | 20 | C-4 | 20 | D-1/D-2 | 4800/2000 |
| Example 10 | R-10 | A-1 | 100 | B-1 | 20 | C-5 | 20 | D-1/D-2 | 4800/2000 |
| Example 11 | R-11 | A-1 | 100 | B-1 | 20 | C-6 | 20 | D-1/D-2 | 4800/7000 |
| Example 12 | R-12 | A-1 | 100 | B-1 | 20 | C-1 | 10 | D-1/D-2 | 4800/7000 |
| Example 13 | R-13 | A-1 | 100 | B-1 | 20 | C-1 | 7 | D-1/D-2 | 4800/2000 |
| Example 14 | R-14 | A-7 | 100 | B-1 | 20 | C-1 | 20 | D-1/D-2 | 4600/2000 |
| Example 15 | R-15 | A-3 | 100 | B-1 | 20 | C-1 | 20 | D-1/D-2 | 4800/2000 |
| Example 16 | R-16 | A-4 | 100 | B-1 | 20 | C-1 | 20 | D-1/D-2 | 4800/2000 |
| Example 17 | R-17 | A-5 | 100 | B-1 | 20 | C-1 | 20 | D-1/D-2 | 4800/2000 |
| Example 18 | R-18 | A-6 | 100 | B-1 | 20 | C-1 | 20 | D-1/D-2 | 4800/7000 |
| Example 19 | R-19 | A-7 | 700 | B-1 | 20 | C-1 | 20 | D-1/D-2 | 4800/2000 |
| Example 20 | R-20 | A-8 | 100 | B-1 | 20 | C-1 | 20 | D-1/D-2 | 4800/2000 |
| Example 21 | R-21 | A-9 | 100 | B-1 | 20 | C-1 | 20 | D-1/D-2 | 4600/2000 |
| Comparative | CR-1 | A-1 | 100 | CB-1 | 20 | C-1 | 20 | D-1/D-2 | 4800/2000 |
| Example 1 | | | | | | | | | |

### <Formation of Resist Pattern (1)>(EUV Exposure, Alkaline Development)

Each of the radiation-sensitive resin compositions prepared above was applied using a spin coater (CLEAN TRACK ACT12 manufactured by Tokyo Electron Ltd.) onto the surface of a 12-inch silicon wafer having a lower layer film with a thickness of 20 nm (AL412 (manufactured by Brewer Science)). After PB was performed at 130° C. for 60 seconds, cooling was performed at 23° C. for 30 seconds to form a resist film having a thickness of 50 nm. Then, the resist film was irradiated with EUV light using an EUV scanner (type “NXE3300”, manufactured by ASML, NA=0.33, lighting condition: Conventional, s=0.89, Mask imecDEFECT 32 FFR 02). The resist film was subjected to PEB at 130° C. for 60 seconds. Then, the resist film was developed at 23° C. for 30 seconds with a 2.38 wt % aqueous TMAH solution to form a 32 nm positive line-and-space pattern.

### <Evaluation>

The sensitivity, depth of focus, and process window of each of the radiation-sensitive resin compositions were evaluated by measuring each of the formed resist patterns according to the following method. A scanning electron microscope (“CG-4100” manufactured by Hitachi High-Tech Corporation) was used for measuring the length of the resist pattern. The evaluation results are shown in the following Table 4.

### [Sensitivity]

An exposure amount at which a 32 nm line-and-space pattern was formed in the formation of the resist pattern (1) was defined as an optimum exposure amount, and the optimum exposure amount was defined as sensitivity (mJ/cm²). Sensitivity of 30 mJ/cm² or less can be evaluated as “excellent”, and sensitivity of more than 30 mJ/cm² can be evaluated as “poor”.

### [Depth of Focus]

In the resist pattern resolved at the optimum exposure amount, the dimension thereof was observed while the focus was changed in the depth direction. A margin in the depth direction was measured, this margin permitting the pattern dimension to be within 90 to 110% of a standard of the dimension without generating any bridge or residue. This measurement result was defined as the depth of focus. The depth of focus of more than 50 nm can be evaluated as good, and the depth of focus of 50 nm or less can be evaluated as poor.

### [Process Window]

A pattern was formed at a low exposure amount to a high exposure amount using a mask forming a line-and-space of 32 nm (1L/1S). In general, connection between patterns is observed on a low exposure amount side, and defects such as pattern collapse are observed on a high exposure amount side. The difference between the upper limit value and the lower limit value of the resist dimension in which these defects were not observed was defined as “CD margin”, and the CD margin of 30 nm or more was determined as good, and the CD margin of less than 30 nm was determined as poor. It is considered that the larger the value of the CD margin is, the wider the process window is.

TABLE 4

| | Radiation- | | | |
|---|---|---|---|---|
| | sensitive | | Depth | CD |
| | resin | Sensitivity | of focus | margin |
| | composition | (mJ/cm²) | (nm) | (nm) |
| Example 1 | R-1 | 27 | 80 | 44 |
| Example 2 | R-2 | 26 | 100 | 38 |
| Example 3 | R-3 | 28 | 80 | 39 |
| Example 4 | R-4 | 26 | 80 | 41 |
| Example 5 | R-5 | 27 | 100 | 43 |
| Example 6 | R-6 | 25 | 60 | 45 |
| Example 7 | R-7 | 26 | 100 | 45 |
| Example 8 | R-8 | 26 | 80 | 44 |
| Example 9 | R-9 | 25 | 60 | 43 |
| Example 10 | R-10 | 26 | 80 | 45 |
| Example 11 | R-11 | 25 | 100 | 48 |
| Example 12 | R-12 | 28 | 80 | 42 |
| Example 13 | R-13 | 29 | 60 | 37 |
| Example 14 | R-14 | 28 | 80 | 47 |
| Example 15 | R-15 | 28 | 80 | 46 |
| Example 16 | R-16 | 29 | 80 | 48 |
| Example 17 | R-17 | 27 | 60 | 45 |
| Example 18 | R-18 | 28 | 60 | 46 |
| Example 19 | R-19 | 29 | 60 | 43 |
| Example 20 | R-20 | 26 | 100 | 46 |
| Example 21 | R-21 | 25 | 100 | 48 |
| Comparative | CR-1 | 42 | 40 | 20 |
| Example 1 | | | | |

As is apparent from the results in Table 4, in all of the radiation-sensitive resin compositions of Examples, the sensitivity, the depth of focus, and the process window (process margins) were better than those of the radiation-sensitive resin composition of Comparative Example.

### Reference Example 1

100 parts by mass of (A-10) as the resin, 12 parts by mass of (C-1) as the radiation-sensitive acid generator, 20 mol % of (B-1) as an acid diffusion controlling agent with respect to (C-1), 2,240 parts by mass of (D-1), 960 parts by mass of (D-3) and 30 parts by mass of (D-4) as the solvent were mixed and the mixture was filtered through a 0.2 μm membrane filter to prepare a radiation-sensitive resin composition (R-22).

### Reference Examples 2 to 6

Radiation-sensitive resin compositions (R-23) to (R-24) and (CR-1) to (CR-3) were prepared in the same manner as in Reference Example 1 except that components of types and contents shown in the following Table 5 were used.

TABLE 5

| | Radiation- | | | Compound (B) | | Radiation-sensitive | | | |
|---|---|---|---|---|---|---|---|---|---|
| | sensitive | Resin (A) | | | with respect | acid generator (C) | | Solvent (D) | |
| | resin | | Parts by | | to (C) | | Parts by | | Parts by |
| | composition | Type | mass | Type | mol % | Type | mass | Solvent (D) | mass |
| Reference | R-22 | A-10 | 100 | B-1 | 20 | C-1 | 12 | D-1/D-3/D-4 | 2240/960/30 |
| Example 1 | | | | | | | | | |
| Reference | R-23 | A-11 | 100 | B-1 | 20 | C-1 | 12 | D-1/D-3/D-4 | 2240/960/30 |
| Example 2 | | | | | | | | | |
| Reference | R-24 | A-12 | 100 | B-1 | 20 | C-1 | 12 | D-1/D-3/D-4 | 2240/960/30 |
| Example 3 | | | | | | | | | |
| Reference | CR-2 | A-10 | 100 | B-1 | 20 | C-1 | 7 | D-1/D-3/D-4 | 2240/960/30 |
| Example 4 | | | | | | | | | |
| Reference | CR-3 | A-11 | 100 | B-1 | 20 | C-1 | 7 | D-1/D-3/D-4 | 2240/960/30 |
| Example 5 | | | | | | | | | |
| Reference | CR-4 | A-12 | 100 | B-1 | 20 | C-1 | 7 | D-1/D-3/D-4 | 2240/960/30 |
| Example 6 | | | | | | | | | |

### <Formation of Resist Pattern (2)>(ArF Exposure, Alkali Development)

To a surface of a 12 inch silicon wafer was applied an underlayer antireflection film forming composition (“ARC66” manufactured by Brewer Science Incorporated.) by using a spin coater (“CLEAN TRACK ACT12” manufactured by Tokyo Electron Limited.). The wafer was then heated at 205° C. for 60 seconds to form an underlayer antireflection film having a film thickness of 105 nm. Each radiation-sensitive resin composition was applied onto the underlayer antireflection film by using the spin coater. PB was performed at 100° C. for 50 seconds. Cooling was then performed at 23° C. for 30 seconds to form a resist film having a film thickness of 90 nm. Next, the resist film was exposed through a mask pattern for forming a resist pattern having a 38 nm line-and-space (1L/1S) with an ArF excimer laser immersion exposure apparatus (“TWINSCAN XT-1900i” manufactured by ASML) in an optical condition of NA=1.35 and Dipole35X (σ=0.97/0.77). After exposing, PEB was performed at 90° C. for 50 seconds. The resist film was subjected to a paddle development by using a 2.38% by mass aqueous TMAH solution at 23° C. for 30 seconds, and then rinsed with ultrapure water for 7 seconds. The resist film was then spin dried at 2,000 rpm for 15 seconds with spinning off to form a resist pattern having a 40 nm line-and-space (1L/1S).

### <Evaluation>

The sensitivity, CDU, and LWR of each of the radiation-sensitive resin compositions were evaluated by measuring each of the formed resist patterns according to the following method. A scanning electron microscope (“CG-5000” manufactured by Hitachi High-Tech Corporation) was used for measuring the length of the resist pattern. The evaluation results are shown in Table 6 below.

### [Sensitivity]

In the formation of the resist pattern (2), an exposure amount for forming a line with a line width of 40 nm formed through a mask pattern for forming a pattern having a 40 nm line-and-space as a target dimension was defined as an optimum exposure amount (Eop).

### [CDU Performance]

A hole pattern formed by exposing to the exposed amount as same as the Eop obtained above was observed from the top of the pattern by using the scanning electron microscope. The hole diameter was measured at 16 points within a square 400 nm on a side, and the measurement values were averaged to determine the average value. The average value was measured at five hundred of optional points. The 3 sigma value was calculated from the distribution of the measurement values, and the 3 sigma value was defined as CDU performance (nm). The smaller the value of CDU performance is, the smaller the variation in the hole diameter over long period is, which is better. As the CDU performance, if the value was 6.0 nm or less, it was evaluated as “good”. If the value was more than 6.0 nm, it was evaluated as “poor”.

### [LWR Performance]

The line-and-space pattern formed by exposing to the exposure amount as same as the Eop calculated in the Formation of Resist Pattern (2) was observed from the top of the pattern by using the scanning electron microscope. The variation in the line width was measured at a total of 500 points. The 3 sigma value was obtained from the distribution of the measurement values, and the 3 sigma value was defined as LWR performance (nm). The smaller the value of the LWR performance is, the smaller the wobble of the line is, which is better. As the LWR performance, if the value was 4.0 nm or less, it was evaluated as “good”. If the value was more than 4.0 nm, it was evaluated as “poor”.

TABLE 6

| | Radiation- | | | |
|---|---|---|---|---|
| | sensitive | | | |
| | resin | Eop | CDU | LWR |
| | composition | (mJ/cm²) | (nm) | (nm) |
| Reference | R-22 | 23 | 5.8 | 3.4 |
| Example 1 | | | | |
| Reference | R-23 | 22 | 5.8 | 3.5 |
| Example 2 | | | | |
| Reference | R- 24 | 21 | 5.7 | 3.3 |
| Example 3 | | | | |
| Reference | CR-2 | 28 | 6.8 | 4.2 |
| Example 4 | | | | |
| Reference | CR-3 | 27 | 6.5 | 4.5 |
| Example 5 | | | | |
| Reference | CR-4 | 26 | 6.7 | 4.3 |
| Example 6 | | | | |

As is apparent from the results in Table 6 above, the radiation-sensitive resin compositions of Reference Examples 1 to 3 had good sensitivity, CDU performance, and LWR performance.

According to the radiation-sensitive resin composition and the method for forming a resist pattern of the embodiments of the present invention, the sensitivity, the depth of focus, and the process margins can be improved as compared with the related art. Therefore, these can be suitably used for forming a fine resist pattern in a lithography process of various electronic devices such as a semiconductor device and a liquid crystal device.

Obviously, numerous modifications and variations of the present invention are possible in light of the above teachings. It is therefore to be understood that within the scope of the appended claims, the invention may be practiced otherwise than as specifically described herein.

What is claimed is:

1. A radiation-sensitive resin composition comprising:
a resin comprising a structural unit having a phenolic hydroxyl group; and
a compound represented by formula (1): 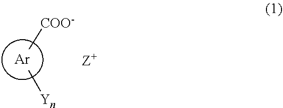
wherein, in the formula (1): Ar is a substituted or unsubstituted aromatic ring having 6 to 20 carbon atoms; n is an integer of 2 to 4; Z⁺ is a monovalent onium cation; a plurality of Ys are each independently a polar group; and at least one of the plurality of Ys is an —OH group or an —SH group bonded to a carbon atom adjacent to a carbon atom to which a COO⁻ group is bonded.

2. The radiation-sensitive resin composition according to claim 1, wherein the polar group bonded to the carbon atom adjacent to the carbon atom to which the COO⁻ group is bonded is an —OH group.

3. The radiation-sensitive resin composition according to claim 1, wherein the compound represented by the formula (1) is represented by formula (1-1): 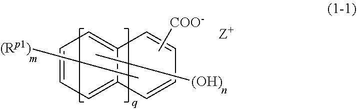
wherein in the formula (1-1): R^{p1} is an alkoxy group, an alkoxycarbonyl group, a halogen atom, or an amino group; m is an integer of 0 to 3; when m is 2 or 3, a plurality of R^{p1}s are the same or different; n and Z⁺ have the same meanings as those in the formula (1); q is an integer of 0 to 2; when q is 0, m+n is less than or equal to 5; and at least one OH group is bonded to the carbon atom adjacent to the carbon atom to which the COO⁻ group is bonded.

4. The radiation-sensitive resin composition according to claim 3, wherein q in the formula (1-1) is 0 or 1.

5. The radiation-sensitive resin composition according to claim 3, wherein n in the formula (1-1) is 2 or 3.

6. The radiation-sensitive resin composition according to claim 1, wherein the onium cation in the formula (1) is a sulfonium cation or an iodonium cation.

7. The radiation-sensitive resin composition according to claim 1, further comprising a radiation-sensitive acid generator which generates an acid having a pKa smaller than that of an acid generated from the compound represented by the formula (1).

8. The radiation-sensitive resin composition according to claim 7, wherein a content of the radiation-sensitive acid generator is 10 parts by mass or more with respect to 100 parts by mass of the resin.

9. The radiation-sensitive resin composition according to claim 8, wherein the content of the radiation-sensitive acid generator is 10 parts by mass or more and 60 parts by mass or less with respect to 100 parts by mass of the resin.

10. The radiation-sensitive resin composition according to claim 8, wherein a molar ratio of a content of the compound represented by the formula (1) to the content of the radiation-sensitive acid generator is 3 mol % or more and 250 mol % or less.

11. The radiation-sensitive resin composition according to claim 1, wherein the structural unit having the phenolic hydroxyl group is derived from hydroxystyrene.

12. The radiation-sensitive resin composition according to claim 1, wherein a content of the structural unit having the phenolic hydroxyl group in the resin is 5 mol % or more and 70 mol % or less.

13. A method for forming a resist pattern comprising:
forming a resist film directly or indirectly on a substrate by applying the radiation-sensitive resin composition according to claim 1;
exposing the resist film; and
developing the exposed resist film.

14. The method for forming a resist pattern according to claim 13, wherein the exposure is performed using extreme ultraviolet ray or electron beam.

15. A radiation-sensitive resin composition comprising:
a resin comprising a structural unit having an acid-dissociable group and not comprising a structural unit having a phenolic hydroxyl group;
a compound represented by formula (1); and
a radiation-sensitive acid generator which generates an acid having a pKa smaller than that of an acid generated from the compound,
wherein a content of the radiation-sensitive acid generator is 10 parts by mass or more with respect to 100 parts by mass of the resin: 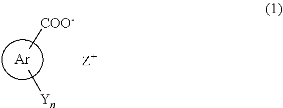
wherein, in the formula (1): Ar is a substituted or unsubstituted aromatic ring having 6 to 20 carbon atoms; n is an integer of 2 to 4; Z⁺ is a monovalent onium cation; a plurality of Ys are each independently a polar group; and at least one of the plurality of Ys is an —OH group or an —SH group bonded to a carbon atom adjacent to a carbon atom to which a COO⁻ group is bonded.

16. The radiation-sensitive resin composition according to claim 15, wherein the compound represented by the formula (1) is represented by formula (1-1): 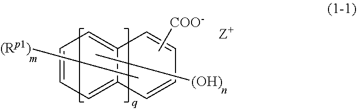
wherein, in the formula (1-1): R^{p1} is an alkoxy group, an alkoxycarbonyl group, a halogen atom, or an amino group; m is an integer of 0 to 3; when m is 2 or 3, a plurality of R^{p1}s are the same or different; n and Z⁺ have the same meanings as those in the formula (1); q is an integer of 0 to 2; when q is 0, m+n is less than or equal to 5; and at least one OH group is bonded to the carbon atom adjacent to the carbon atom to which the COO⁻ group is bonded.

17. The radiation-sensitive resin composition according to claim 16, wherein q in the formula (1-1) is 0 or 1.

18. The radiation-sensitive resin composition according to claim 16, wherein n in the formula (1-1) is 2 or 3.

19. A method for forming a resist pattern comprising:
forming a resist film directly or indirectly on a substrate by applying the radiation-sensitive resin composition according to claim 15;
exposing the resist film; and
developing the exposed resist film.

20. The method for forming a resist pattern according to claim 19, wherein the exposure is performed using extreme ultraviolet ray or electron beam.
